SEQUENCE LISTING (4 sequences)

SEQ ID NO: 1 - DNA, 19 bp, Artificial sequence
  Synthetic oligonucleotide
atgccgcccgcctgtcctt

SEQ ID NO: 2 - DNA, 22 bp, Artificial sequence
  Synthetic oligonucleotide
gggtccttgagcctcatcatca

SEQ ID NO: 3 - DNA, 23 bp, Artificial sequence
  Synthetic oligonucleotide
ggtgcgcgtcctgctgctctgta

SEQ ID NO: 4 - DNA, 23 bp, Artificial sequence
  Synthetic oligonucleotide
agtggcgtgttcccgtgctctcc

(19) United States
(12) Patent Application Publication
(10) Pub. No.: US 2010/0048727 A1
(43) Pub. Date: Feb. 25, 2010

(54) PERFLUOROKETONE COMPOUNDS AND USES THEREOF

(76) Inventors: Samuel David, Dorval (CA); Athena Kalyvas, Laval (CA); Ruben Lopez-Vales, Montreal (CA); George Kokotos, Athens (GR); Violetta Constantinou-Kokotou, Athens (GR); Constantinos Baskakis, Athens (GR); Christoforos G. Kokotos, Athens (GR); Edward A. Dennis, La Jolla, CA (US); Daren Stephens, Raleigh, NC (US)

Correspondence Address: KLARQUIST SPARKMAN, LLP, 121 SW SALMON STREET, SUITE 1600, PORTLAND, OR, 97204, US

(21) Appl. No.: 12/594,579

(22) PCT Filed: Apr. 4, 2008
(86) PCT No.: PCT/CA2008/000638

§ 371 Date: Oct. 2, 2009

Related U.S. Application Data
(60) Provisional application No. 60/910,302, filed on Apr. 5, 2007.

(51) Int. Cl. A61K 31/121; C12N 5/00; A61K 31/12; C07C 49/11; C07C 45/54; C07C 49/105; C07C 49/115; C07C 49/04; A61P 25/00; A61P 29/00

(52) U.S. Cl. 514/679; 435/375; 514/675; 514/678; 514/682; 568/308; 568/316; 568/319; 568/325; 568/328; 568/419

(57) ABSTRACT

Novel perfluoroketone compounds of formula [I] and [Ia] are described. Also described are uses thereof, such as for inhibition of phospholipase A₂ activity. Therapeutic uses thereof are also described, such as for the treatment of neural conditions and/or inflammatory conditions, such as demyelinating (e.g., multiple sclerosis) and neural injury (e.g., spinal cord injury).

### CROSS-REFERENCE TO RELATED APPLICATIONS

This application claims the benefit, under 35 U.S.C. §119(e), of U.S. provisional application Ser. No. 60/910,302 filed on Apr. 5, 2007, which is incorporated herein by reference in its entirety.

### FIELD OF THE INVENTION

The present invention relates to perfluoroketone compounds as well as salts, hydrates and derivatives thereof, and compositions containing them. The invention further relates to uses of such compounds, salts, hydrates, derivatives and compositions, such as for the inhibition of phospholipase A₂ and/or the treatment of various conditions (e.g., neural and/or inflammatory conditions).

### BACKGROUND OF THE INVENTION

Multiple sclerosis (MS) is a multi-focal autoimmune demyelinating disease of the central nervous system (CNS) that affects over a million people worldwide (Compston, A. and Coles, A. (2002). Lancet 359: 1221-31; Noseworthy, J. et al. (2000). N. Engl. J. Med. 343: 938-52; Steinman, L. et al. (2002). Annu. Rev. Neurosci. 25: 491-505; Hemmer, B. et al. (2002), Nature Rev. Neurosci. 3: 291-301). The disease onset is generally between the second and third decades of life, affecting women more often than men. Increasing cases of juvenile disease are also being reported. The clinical symptoms are varied and include motor paralysis, sensory loss or paresthesias, and bowel and bladder dysfunction, as the focal inflammatory lesions can occur in any region of the CNS. Although the cause of MS is still not fully known, genetic and environmental factors increase susceptibility to the disease (Noseworthy, J. et al., supra).

Experimental autoimmune encephalomyelitis (EAE) is a widely used rodent model of MS (Owens, T. and Sriram, S. (1995). Neurol. Clin. 13: 51-73; Steinman, L. (1999). Neuron. 24: 511-4). This animal model has provided important insights into the onset and progression of CNS autoimmune demyelinating disease. In the EAE model in which mice are immunized with myelin antigens mixed with adjuvant (Owens, T. and Sriram, S., supra; Steinman, L., supra; Johns, T. G. et al. (1995). J Immunol. 154: 5536-41), T cells become activated in the periphery to a Th1 phenotype (as reflected for example in interferon-γ and IL-2 expression), then migrate to the CNS where the myelin-reactive T cells become reactivated by antigen-presenting cells. The reactivated Th1 T cells induce the further recruitment of T cells and macrophages and activation of CNS glia (microglia and astrocytes), which then leads to demyelination and axonal damage. EAE shares some of the pathological features of MS and has helped to understand some of the complex immunological networks that mediate disease.

Although the primary events that trigger MS are still not known, the findings to date suggest that T-cells in the periphery become reactive to certain myelin antigens by as yet unknown mechanisms, eventually resulting in robust inflammatory lesions in the CNS. The exact mechanisms underlying the formation of these lesions in the CNS are not fully understood.

Several experimental approaches have been tested in an effort to ameliorate EAE symptoms. Most of these involve immune modulation. These include treatments to block various chemokines or cytokines and to prevent the actions of macrophages and T cells. Of these efforts to develop new treatments for MS, only a few have been approved and are in use (Steinman, L., supra; Polman and Uitdehaag, (2000) Lancet Neurol. 2 (9): 563-6). MS therapies currently being used consist of immunomodulatory drugs such as corticosteroids, Interferon beta, and Glatiramer acetate.

Corticosteroids have anti-inflammatory and immunosuppressive effects, which also transiently restores the blood-brain barrier (Noseworthy et al., (2000) Neurology 54(9): 1726-33). They shorten the duration of the relapse and accelerate recovery. Since they are only effective as a short-term treatment, they are most commonly used to treat an acute relapse (Andersson and Goodkin, (1998) J Neurol Sci. 160(1): 16-25; Bansil et al., (1995) Ann Neurol. 37 Suppl 1: S87-101). Further, the responsiveness to corticosteroids declines over time, and extended use may lead to adrenal suppression, cardiovascular collapse and arrhythmias. (C. F. Lacy, L. L. Armstrong, M. P. Goldman, L. L. Lance. Drug information handbook 8^{th} Edition, 2001, pp. 549-551).

lnterferon-β has been used as a therapy for patients with active Relapsing/Remitting Multiple Sclerosis (RRMS) since the 1980's. It is recently being used for secondary progressive patients as well. The exact mode of action of this drug is not yet known. It is thought to play an immunomodulatory role by suppressing T cell-mediated inflammation (Stinissen et al., (1997) Crit Rev Immunol. 17(1): 33-75). Recombinant IFN is available in 3 drugs: IFNβ-Ib (Betaseron™) and two IFNβ-Ia preparations (Avonex™ and Rebif™) (Polman and Uitedehaag, supra). These drugs reduce the rate of clinical relapse. However, neutralizing antibodies develop against these drugs rendering them ineffective with time. Also, flu-like symptoms are a prominent side effect early on in the treatment.

Glatiramer acetate (Copaxone™) is: a synthetic co-polymer of tyrosine, glutamate, alanine and lysine, thought to mimic myelin basic protein (MBP) and thus, block T cell recognition of MBP (Karin N. et al., (1994) J Exp Med. 180(6): 2227-37). This drug is therefore beneficial in RRMS but not progressive MS. This drug also decreases the rate of relapse and appears to be better tolerated by patients than interferon therapy. Further, treatment with this drug may cause cardiovascular problems such as chest pain, flushing and tachycardia, and respiratory problems such as dyspnea. (C. F. Lacy, L. L. Armstrong, M. P. Goldman, L. L. Lance. Drug information handbook 8^{th} Edition, 2001, pp. 777-779) Recently, another drug that has been approved for the use in RRMS and secondary progressive MS is mitroxantrone. This drug is used to arrest the cell cycle and prevent cellular division, and it is primarily used in the treatment of leukemia (Rolak L. A., (2001) Neurol Clin. 19(1): 107-18). In MS, it reduces relapse rate and increases the length between exacerbations. This drug however has long-term side effects causing cardiac toxicity. Another treatment is intravenous immunoglobulin. It acts to alter the immune system in a beneficial way and it has shown to cut relapses in half (Rolak, supra). However, the treatments are very expensive.

Therefore, there are a few moderately effective treatments for RRMS and secondary progressive MS that have shown to reduce both the frequency of the disease and severity of exacerbations. However, problems still exist in treating MS, and there are still no proven treatments, for example, for primary progressive MS. There is therefore a continued need for improved materials and methods for the treatment of neurodegenerative diseases such as MS.

Spinal cord injury (SCI) occurs due to traumatic injuries resulting from for example traffic accidents, athletic accidents, or falls and drops from heights, and to spinal cord compression, or the like. It also occurs due to other disorders, for example, when stroke is accompanied by pyramidal tract transection. Spinal cord injury results in permanent loss of motor, sensory and autonomic functions.

Following the initial injury, presumably as part of the inflammatory/immune response to the injury, a series of degenerative processes which promote tissue damage beyond the original site of injury are initiated. After the initial mechanical disruption of nerves and nerve fibers at the time of injury, hemorrhaging is usually observed within 30 minutes at the area of damage and may expand over the next few hours. Within several hours following the injury, inflammatory cells, e.g., neutrophils and macrophages, infiltrate the area and cause further damage to the nerve tissue, i.e., cell-mediated damage. These post-traumatic events are referred to as “secondary injury” (or “secondary spinal cord injury”). Therefore, a significant aspect of the tissue damage and functional loss may be preventable as it is the result of secondary events triggered by the trauma. It is important to treat as promptly as possible when the spinal cord is damaged, in order to promote recovery from or to prevent progress, of neurologic function deficit. It would be advantageous to prevent further damage to the spinal cord and surrounding tissue following a spinal cord injury by treatment as soon as possible after the initial trauma to prevent secondary injury effects.

Currently, the conventional treatment for reducing or minimizing the damage resulting from secondary injury is intravenous injection of the glucocorticoid, methylprednisolone (Bracken et al., JAMA, 277(20): 1597-1604 (1997)). Unfortunately, prolonged administration of glucocorticoids has adverse systemic side effects, e.g., increased incidence of sepsis and pneumonia, and a limited therapeutic window.

Therefore, there is also a need for novel approaches and treatments for neural injury, such as spinal cord injury.

The present description refers to a number of documents, the content of which is herein incorporated by reference in their entirety.

### SUMMARY OF THE INVENTION

The present invention relates to perfluoroketone compounds as well as salts, hydrates and derivatives thereof, and compositions containing them. The invention further relates to uses of such compounds, salts, hydrates, derivatives and compositions, such as for the inhibition of phospholipase A₂ and/or the treatment of various conditions (e.g., neural and/or inflammatory conditions).

In a first aspect, the present invention provides perfluoroketone compounds having the formula I, and hydrates thereof having the formula Ia:

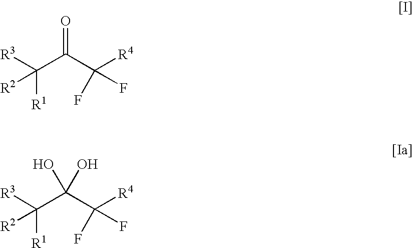

wherein;
R¹ is H, F or CH₃;
R²is H or F;
R³ is alkyl, branched or linear, saturated or unsaturated; aryl, substituted or not; or
heteroaryl, substituted or not;

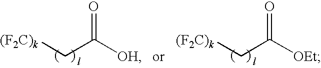

R⁴ is H, F, CF₃, (CF₂)ₖCF₃,
wherein k=0-4and l=2-5,
   or a pharmaceutically acceptable salt thereof.

In an embodiment, R³ is:

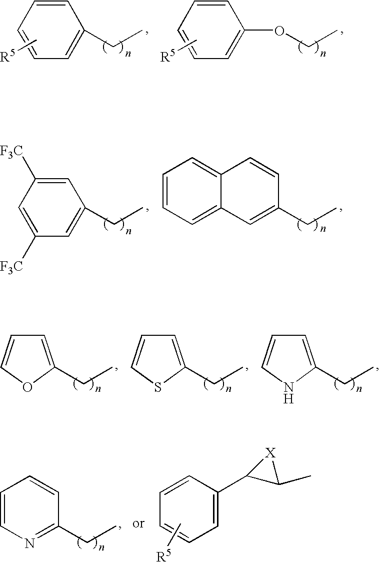

wherein;
X=0, NH or S;
n=1-5

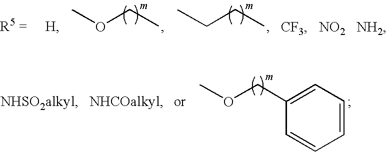

wherein m=1-9.

In an embodiment, the above-mentioned alkyl is C₁-C₁₄ linear alkyl.

In an embodiment, the above-mentioned alkyl is C₆-C₁₂ linear alkyl.

In an embodiment, the above-mentioned aromatic group is a 1 or 2 ring aromatic group.

In an embodiment, R⁴ is F.

In an embodiment, R⁴ is CF₃.

In an embodiment, R⁴ is C₂F₅.

In an embodiment, R⁴ is F and R¹ is F.

In an embodiment, R⁴ is F, R¹ is F and R² is F.

In further embodiments, n=1, 2, 3, 4 or 5.

In an embodiment, the above-mentioned compound is:

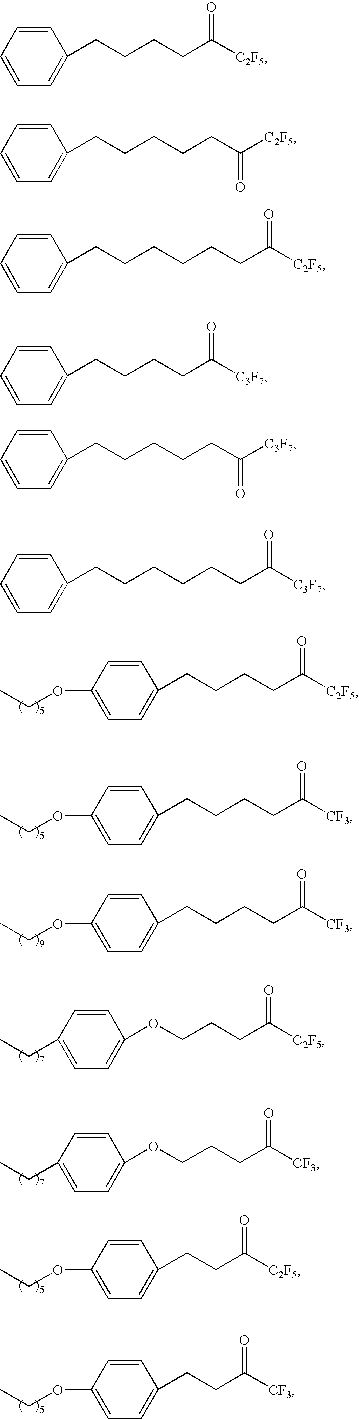

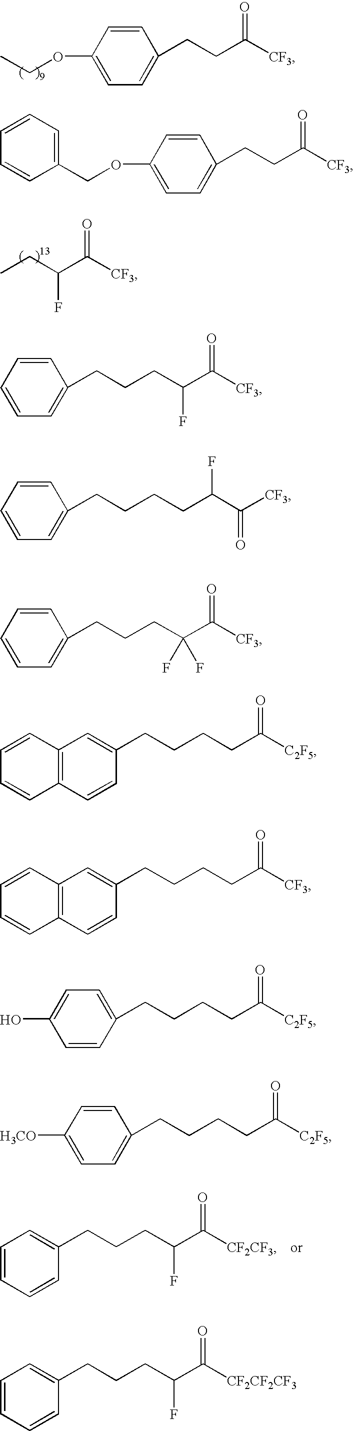

or a hydrate thereof, or a salt thereof.

In a further embodiment, the above-mentioned compound is

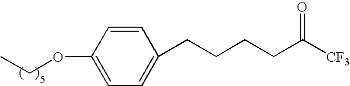

In another embodiment, the above-mentioned compound is

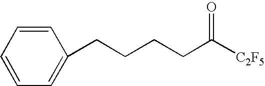

In another aspect, the present invention provides a composition comprising the above-mentioned compound and a pharmaceutically acceptable carrier or excipient.

In another aspect, the present invention provides a use of the above-mentioned compound as a medicament.

In another aspect, the present invention provides a use of the above-mentioned compound for the preparation of a medicament.

In another aspect, the present invention provides a method for inhibiting PLA₂ activity in a system (e.g., a cell-free system), cell or subject, said method comprising contacting said system or cell with, or administering to said subject, an effective amount of the above-mentioned compound or composition.

In another aspect, the present invention provides a method for the preventing and/or treating an inflammatory disease or condition in a subject, said method comprising administering to said subject an effective amount of the above-mentioned compound or composition.

In another aspect, the present invention provides the use of the above-mentioned compound or composition for inhibiting PLA₂ activity in a system (e.g., a cell-free system), cell or subject.

In another aspect, the present invention provides the use of the above-mentioned compound or composition for the preparation of a medicament for inhibiting PLA₂ activity in a cell or subject.

In another aspect, the present invention provides the use of the above-mentioned compound or composition for the prevention and/or treatment of an inflammatory disease or condition.

In another aspect, the present invention provides the use of the above-mentioned compound or composition for the preparation of a medicament for the prevention and/or treatment of a neural disease or condition.

In another aspect, the present invention provides the above-mentioned compound or composition for use in the inhibition of PLA₂ activity in a cell or subject.

In another aspect, the present invention provides the above-mentioned compound or composition for use in the prevention and/or treatment of a neural disease or condition.

In another aspect, the present invention provides the above-mentioned compound or composition for use in the prevention and/or treatment of an inflammatory disease or condition.

In an embodiment, the above-mentioned inflammatory disease or condition is a neural disease or condition. In a further embodiment, the above-mentioned neural disease or condition is an inflammatory disease or condition of the central nervous system (CNS). In a further embodiment the above-mentioned neural disease or condition is a non-CNS inflammatory disease or condition.

In an embodiment, the above-mentioned disease or condition is a neural injury. In a further embodiment, the above-mentioned neural injury is spinal cord injury (SCI).

In yet another embodiment, the above-mentioned neural disease or condition is a demyelinating disease. In a further embodiment, the above-mentioned demyelinating disease is Multiple Sclerosis (MS).

In a further embodiment, the above-mentioned neural disease or condition or inflammation is associated with PLA₂ activity. In a further embodiment, the above-mentioned PLA₂ activity is iPLA₂ activity. In a further embodiment, the above-mentioned iPLA₂ activity is group VIA iPLA₂ activity.

In an embodiment, the above-mentioned subject is a mammal. In a further embodiment, the above-mentioned mammal is a human.

In another aspect, the present invention provides a package or kit comprising the above-mentioned compound or composition together with instructions for the prevention or treatment of a neural disease or condition.

In another aspect, the present invention provides a method of preparing a perfluoroketone compound of the invention. In an embodiment, a perfluoroketone compound of the present invention can be prepared from a carboxylic acid by conversion to the corresponding acyl chloride or fluoride and treatment with the anhydride of the appropriate perfluoro acid in the presence of an amine. In another embodiment, pentafluoroethyl and heptafluoropropyl ketones of the present invention can be prepared from N-methoxy-N-methyl amides of carboxylic acids or symmetric anhydrides of carboxylic acids or morpholino amides of carboxylic acids by treatment of each one with CF₃CF₂I or CF₃CF₂CF₂I followed by treatment with an organo-lithium reagent (e.g., MeLi.LiBr). Also, from aldehydes by treatment with CF₃CF₂I or CF₃CF₂CF₂I followed by treatment with an organo-lithium reagent (e.g., MeLi.LiBr); then, the secondary alcohol is oxidized to pentafluoroethyl or heptafluoropropyl ketone.

In another aspect, the present invention provides a method of preparing the perfluoroketone compound of formula I as defined above, the method comprising:
(a) converting a carboxylic acid of formula II

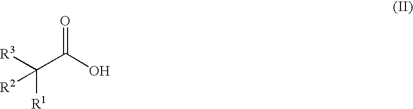

into a corresponding acyl chloride or acyl fluoride of formula III

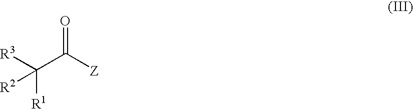

wherein Z═Cl or F; and
(b) reacting the compound of formula III with an anhydride of a perfluoroacid of formula IV

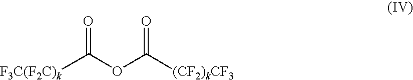

in the presence of an amine,
wherein k, R¹, R² and R³ are as defined above;
thereby obtaining the perfluoroketone compound of formula I.

In another aspect, the present invention provides a method of preparing the perfluoroketone compound of formula I as defined above, the method comprising:
(a) reacting a compound of formula (V), (VI) or (VII)

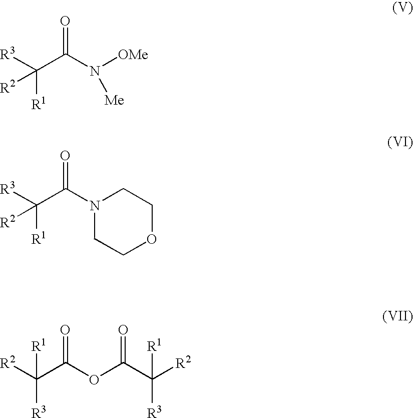

with a compound of formula (VIII)

I(CF₂)ₖCF₃ (VIII)
wherein k, R¹, R² and R³ are as defined above; and
(b) treating the reaction mixture of (a) with an organo-lithium reagent, thereby obtaining the perfluoroketone compound of formula I.

A method of preparing the perfluoroketone compound of formula I as defined above, the method comprising:
(a) reacting an aldehyde of formula IX

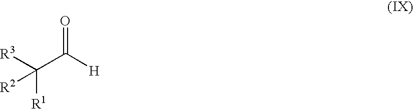

with a compound of formula VIII

I(CF₂)ₖCF₃ (VIII)
(b) treating the reaction mixture of (a) with an organo-lithium reagent to obtain a compound of formula X

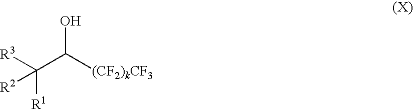

wherein k, R¹, R² and R³ are as defined above; and
(c) oxidizing the compound of formula X;
thereby obtaining the perfluoroketone compound of formula I.

In an embodiment, the above-mentioned organo-lithium reagent is MeLi.LiBr.

In another embodiment, the above-mentioned compound of formula V is obtained by conversion of the above-mentioned carboxylic acid of formula II.

In another embodiment, the above-mentioned reaction with a compound of formula VIII is carried out at a temperature of about −78° C.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows an RT-PCR analysis of mRNA expression of cPLA₂ IVA and iPLA₂ VIA in the spinal cord and spleen of normal mice, and EAE mice at the onset, peak and remission stages;

FIG. 2 shows immunofluorescence micrographs showing cPLA₂⁺ immune cells entering the spinal cord in onset and peak stages of disease. Double labeling of GFAP⁺ astrocytes is observed at peak stage of disease. cPLA₂ expression returns to naïve levels in remission stage;

FIG. 3 shows the expression of cPLA₂ in immune cells at different phases of EAE. Bar chart representing the percentages of immune cell types expressing cPLA₂ at onset (hatched bars), peak (black bars) and remission (grey bars) phases of disease;

FIG. 4 shows immunofluorescence micrographs showing iPLA₂⁺ immune cells entering the spinal cord in onset and peak stages of disease. There is no co-labeling with GFAP⁺ astrocytes in any stage of disease. iPLA₂ expression returns to naïve levels in remission stage;

FIG. 5 shows the expression of iPLA₂ in immune cells at different phases of EAE. Bar chart representing the percentages of immune cell types expressing iPLA₂ at onset (hatched bars), peak (black bars) and remission (grey bars) phases of disease;

FIG. 6 shows the clinical course of SJL/J mice induced with EAE treated (grey squares) or not (black circles) with the specific iPLA₂ inhibitor FKGK11. (A) FKGK11 (or the vehicle) was administered from days 5 to 24 after induction of EAE. Data represent means±s.e.m. from two independent experiments, with a total of 19 mice in each group. (B) FKGK11 (or the vehicle) was administered from the day mice began to show symptoms (day 11) for a 3-week period. Data represent means±s.e.m. from an experiment with a total of 10 mice in each group;

FIG. 7 shows the clinical course of SJL/J mice induced with EAE treated (grey squares) or not (black circles) with the broad PLA₂ inhibitor FKGK2. (A) FKGK2 (or the vehicle) was administered from days 5 to 24 after induction of EAE. Data represent means±s.e.m. from two independent experiments, with a total of 19 mice in each group. (B) FKGK2 (or the vehicle) was administered from the day mice began to show symptoms (day 11) for a 3-week period. Data represent means±s.e.m. from an experiment with a total of 10 mice in each group;

FIG. 8 shows the expression of cytokines (A) and chemokines (B) in spinal cords from EAE mice, in the presence or absence of the specific iPLA₂ inhibitor FKGK11. For each cytokine/chemokine tested: white bar (left)=naïve mice; black bar (center)=vehicle-treated mice; gray bar (right)=FKGK11-treated mice;

FIG. 9 shows the expression of iPLA₂ after spinal cord contusion injury in mice. (A) Quantification of the changes in mRNA levels of iPLA₂ group GVIA from 1 to 28 days after SCI by RT-PCR. Significant up-regulation of iPLA₂ mRNA levels is observed at 14 dpi (p<0.05). (B) Quantification of iPLA₂ GVIA protein levels from 1 to 28 after SCI by Western blotting. The 84 kDa form is significant up-regulated at 14 dpi (*p<0.05). Activated form of iPLA₂ (˜50KDa) is significant up-regulated from 7 to 21 days after SCI, peaking at 14 dpi (p<0.01). (C) Double immunofluorescence images of iPLA₂ GVIA (green) co-labaled with anti-CC-1 (oligodendrocytes), GFAP (astrocytes), anti-Mac-1 (macroglia/macrophages), anti-NeuN (neurons) and SMI312 antibody (axons). Note that iPLA₂ GVIA is mainly expressed in oligodendrocytes and in axons. Note that in the latter, iPLA₂ GVIA is expressed on the axonal membrane of the large SMI312⁺ as well as in smaller SMI312⁻ axons; and

FIG. 10 shows the effect of FKGK11 on various markers of neural damage. The time course of locomotor recovery evaluated using (A) the BMS and (B) locomotor BMS subscores. The BMS subscores typically evaluate finer aspects of locomotor control. Treatment with FKGK11 (black inverted triangles) results in significantly better BMS subscores at 28 days after SCI as compared to untreated mice (*p<0.01) (B). No significant differences were seen in the main BMS scores (A). (C) Treatment with FKGK11 leads to greater amount of tissue sparing at the epicenter and in adjacent areas at 28 days after SCI (*p<0.05). (D) No significant differences were seen in the survival of neurons in the ventral horn in mice treated with FKGK11. (E) Treatment with FKGK11 results in greater sparing of myelin assessed by Luxol fast blue staining, in areas adjacent to the lesion epicenter at 28 days after SCI. (F) Quantification of serotonergic fibers at 1000 μm caudal to the epicenter 28 days after SCI. Mice treated with FKGK11 show significantly greater sparing of serotonergic fibers in the lateral funiculi (*p<0.01). Barr=50 μm.

### DESCRIPTION OF ILLUSTRATIVE EMBODIMENTS

Described herein are studies using mouse model systems of neural diseases/conditions, namely mouse models of Multiple Sclerosis (EAE mice) and spinal cord injury, which show that treatment with novel perfluoroketone compounds significantly decrease inflammation and/or improve the clinical symptoms associated with these diseases.

Accordingly, in a first aspect, the present invention provides perfluoroketone compounds having the formula I, and hydrates thereof having the formula Ia:

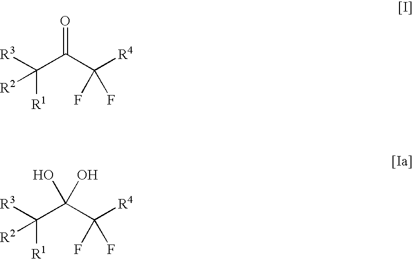

wherein;
R¹ is H, F or CH₃;
R² is H or F;
R³ is alkyl, branched or linear, saturated or unsaturated; aryl, substituted or not; or
heteroaryl, substituted or not;

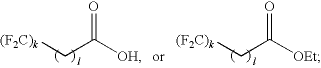

R⁴ is H, F, CF₃, (CF₂)ₖCF₃,
k=0-4 and l=2-5,
   or a pharmaceutically acceptable salt thereof.

In an embodiment, R³ is:

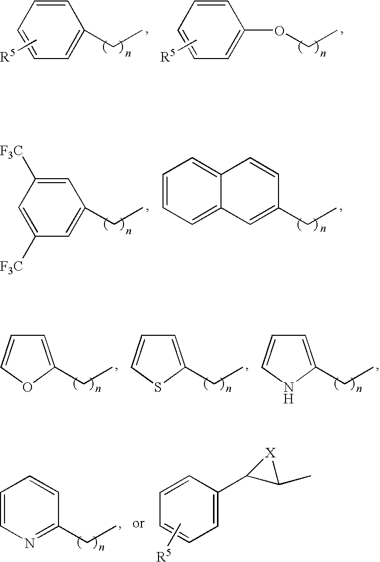

wherein;
X=0, NH or S;
n=1-5

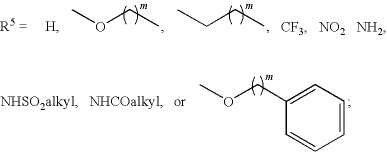

and
m is an integer.

In an embodiment, m=1-9.

In an embodiment, the above-mentioned alkyl is C₁-C₁₄ linear alkyl.

In an embodiment, the above-mentioned alkyl is C₆-C₁₂ linear alkyl.

In an embodiment, the above-mentioned aromatic group is a 1 or 2 ring aromatic group.

In an embodiment, R⁴ is F.

In an embodiment, R⁴ is CF₃.

In an embodiment, R⁴ is C₂F₅.

In an embodiment, R⁴ is F and R¹ is F.

In an embodiment, R⁴ is F, R¹ is F and R² is F.

In further embodiments n=1, 2, 3, 4 or 5.

In an embodiment, the above-mentioned compound is:

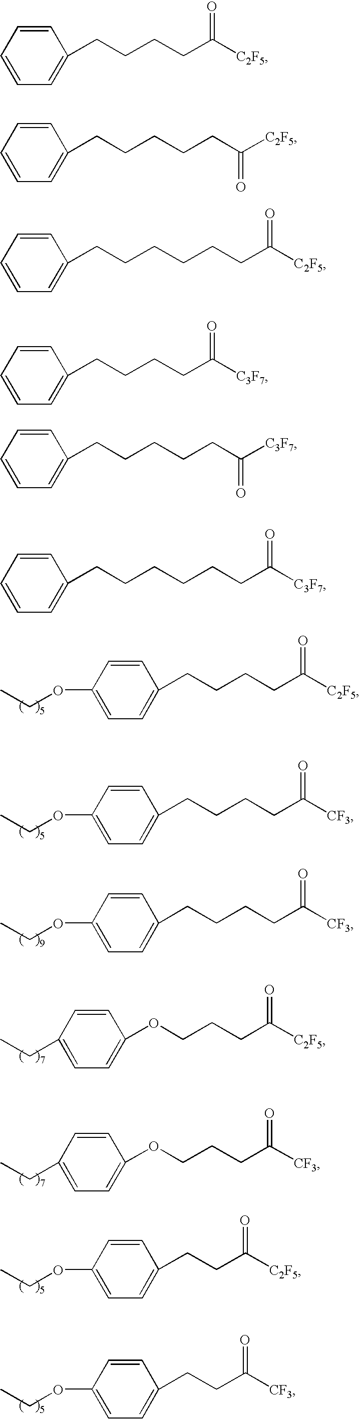

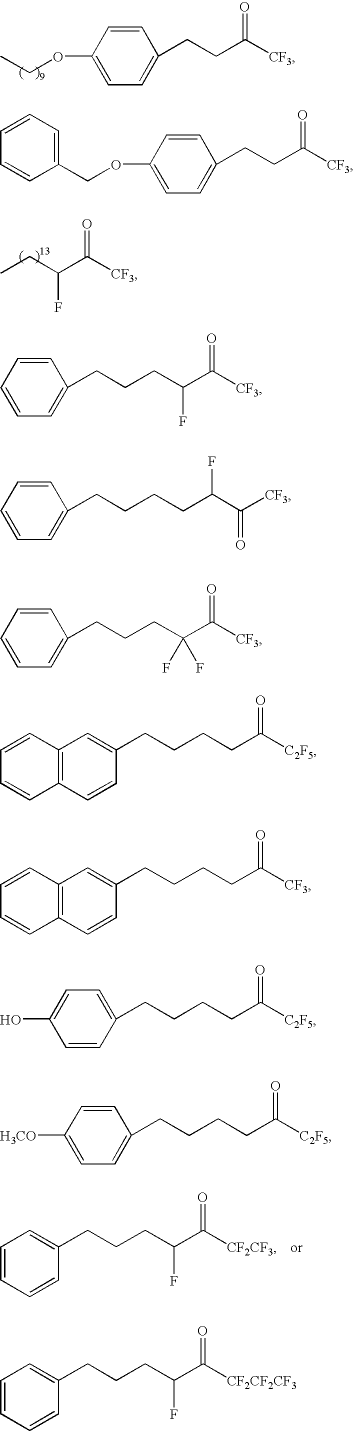

or a hydrate thereof (whereby the carbonyl group is replaced by two hydroxyl groups), or a salt thereof.

In an embodiment, the above-mentioned compound is:

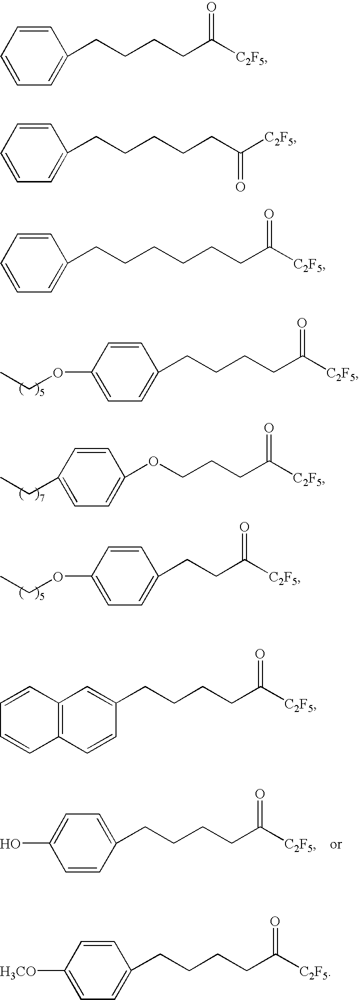

In an embodiment, the above-mentioned compound is:

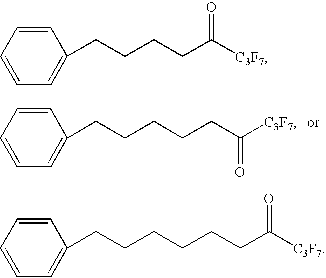

In an embodiment, the above-mentioned compound is:

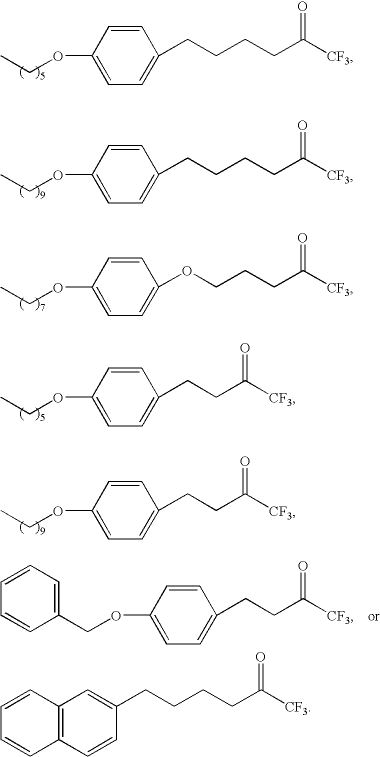

In an embodiment, the above-mentioned compound is:

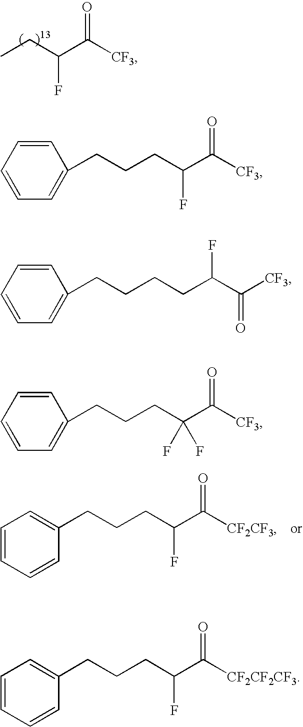

In a further embodiment, the above-mentioned compound is

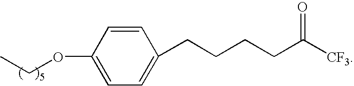

In another embodiment, the above-mentioned compound is

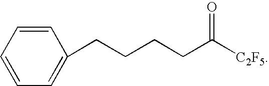

Synthesis of representative perfluoroketone compounds is detailed in Examples 2 to 14 below.

General Routes for the Synthesis of Perfluoroketone Compounds.

Pentafluoroethyl ketones were prepared as depicted in Scheme 1. Trifluoromethyl ketones as well as heptafluoropropyl ketones may be prepared in a similar manner.

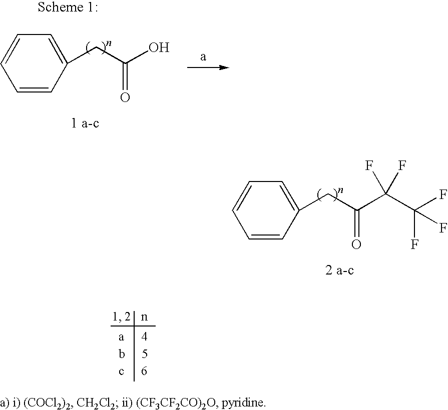

Trifluoromethyl ketones and pentafluoroethyl ketones may be prepared as described in Scheme 2.

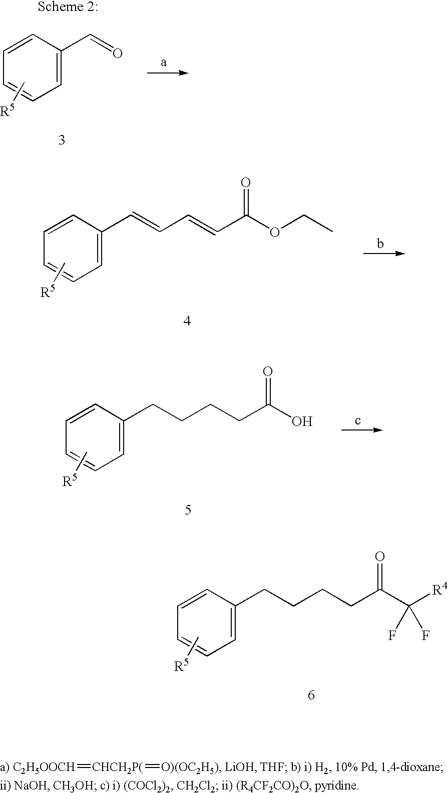

α,α,α-Trifluoromethyl-α′-fluoro ketones may be prepared as described in Scheme 3.

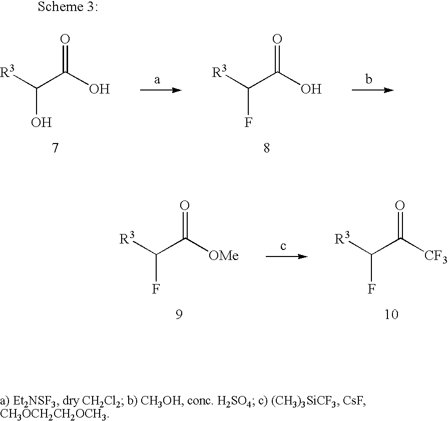

α,α,α-Trifluoromethyl-α′,α′-difluoro ketones may be prepared as described in Schemes 4, 5.

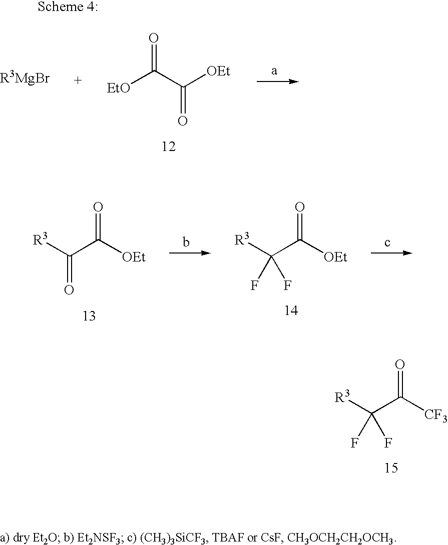

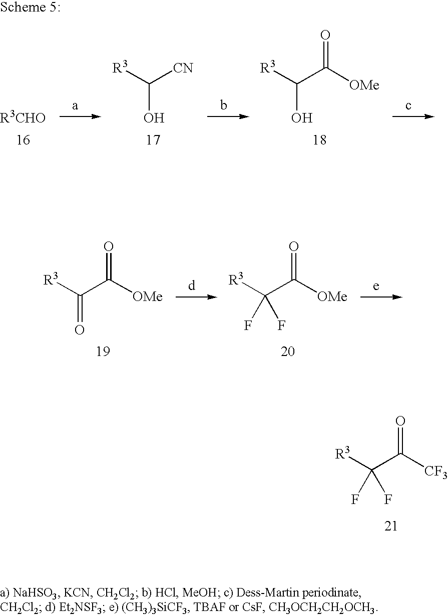

Pentafluoroethyl and heptafluoropropyl ketones may be prepared through the N-methoxy-N-methyl amide, symmetric anhydride and morpholino amide of the appropriate carboxylic acid as depicted in Schemes 6, 7 and 8. In these schemes the synthesis of FKGK11 and FKGK19 is illustrated. Treatment of the N-methoxy-N-methyl amide 22 with CF₃CF₂I or CF₃CF₂CF₂I followed by MeLi.LiBr at −78° C. gave the corresponding fluoroketones in almost quantitative yield. However, in the case of symmetric anhydride 23 or morpholino amide 26, treatment with CF₃CF₂I or CF₃CF₂CF₂I followed by MeLi.LiBr at −78° C. led to mixtures of the desired fluoroketones with fluorinated tertiary alcohols 24 and 25.

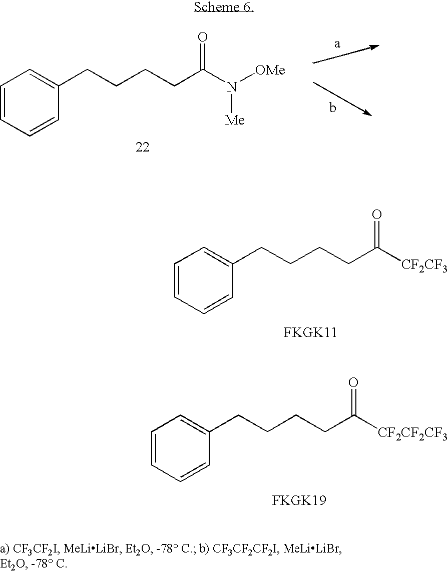

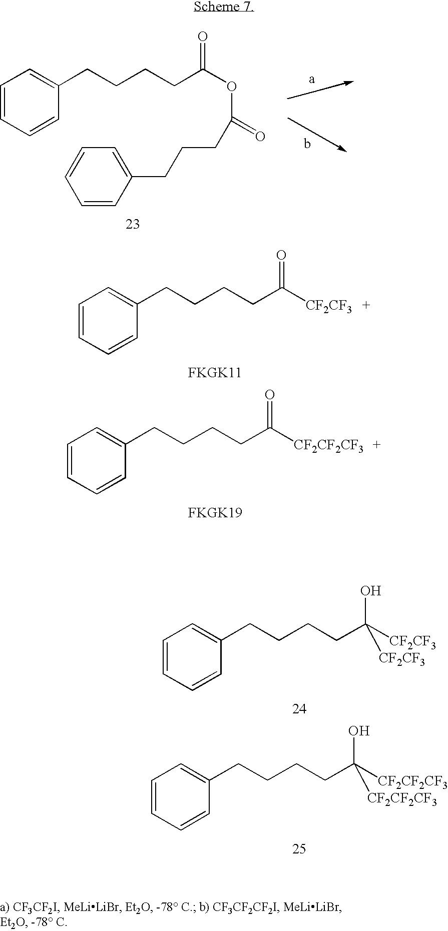

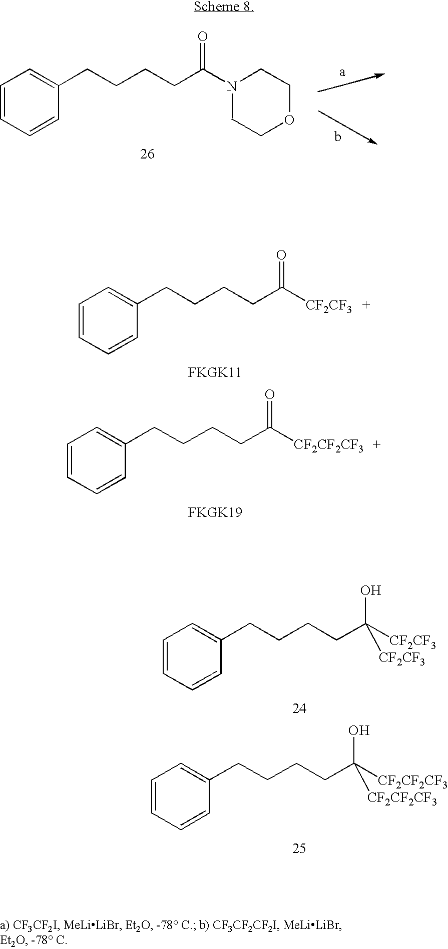

Pentafluoroethyl and heptafluoropropyl ketones may be also prepared from the corresponding aldehyde by treatment with CF₃CF₂I or CF₃CF₂CF₂I followed by MeLi.LiBr at −78° C. Then, the resulting secondary alcohols are oxidized to the target ketones by an oxidative agent, for example Dess-Martin periodinane. In Scheme 9, the synthesis of FKGK11 and FKGK19 is shown.

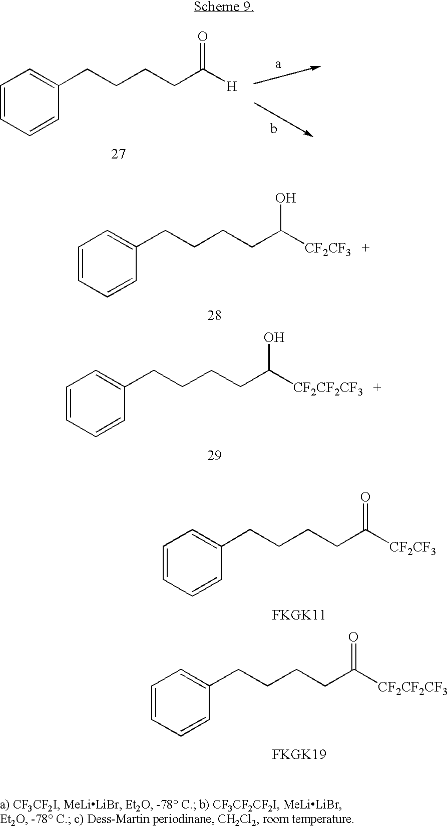

The method based on the treatment of the N-methoxy-N-methyl amide with CF₃CF₂I or CF₃CF₂CF₂I followed by MeLi.LiBr at −78° C. may be used for the synthesis of α′-fluoro pentafluoroethyl and heptafluoropropyl ketones (FKGK21 and FKGK22, Scheme 10).

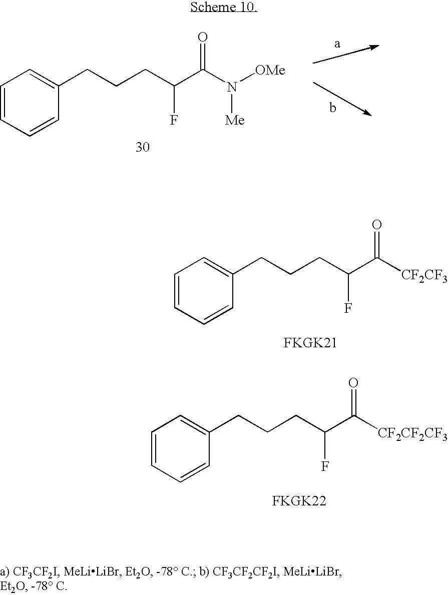

An alternative method that may be used for the synthesis of pentafluoroethyl ketones is the conversion of the carboxylic acid into the corresponding fluoride followed by subsequent treatment with the appropriate anhydride (Scheme 11).

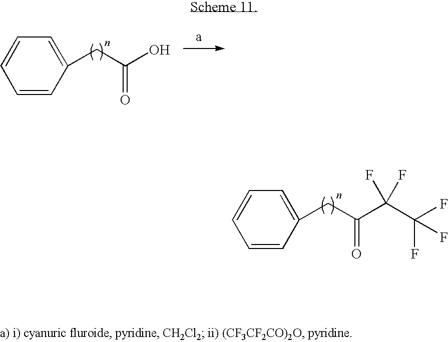

Some of the compounds described herein contain one or more asymmetric centers and may thus give rise to diastereomers and optical isomers. The present invention is meant to include such possible diastereomers as well as their racemic and resolved, enantiomerically pure forms, and pharmaceutically acceptable salts thereof.

The term “alkyl” refers to the radical of saturated aliphatic groups, including straight chain alkyl groups, branched-chain alkyl groups, cycloalkyl(alicyclic) groups, alkyl substituted cycloalkyl groups, and cycloalkyl substituted alkyl groups. Typical alkyl groups include, but are not limited to, methyl, ethyl, propyl, isopropyl, butyl, isobutyl, t-butyl, pentyl, isopentyl, hexyl, etc. The alkyl groups can be (C₁-C₆) alkyl, or (C₁-C₃) alkyl. The term “lower alkyl” refers to alkyl groups having up to 6 carbons (C₁-C₆). A “substituted alkyl” has substituents replacing a hydrogen on one or more carbons of the hydrocarbon backbone. Such substituents can include, for example, halogen, hydroxyl, carbonyl (such as carboxyl, ketones (including alkylcarbonyl and arylcarbonyl groups), and esters (including alkyloxycarbonyl and aryloxycarbonyl groups)), thiocarbonyl, acyloxy, alkoxyl, phosphoryl, phosphonate, phosphinate, amino, acylamino, amido, amidine, imino, cyano, nitro, azido, sulfhydryl, alkylthio, sulfate, sulfonate, sulfamoyl, sulfonamido, heterocyclyl, aralkyl, or an aromatic or heteroaromatic moiety. The moieties substituted on the hydrocarbon chain can themselves be substituted, if appropriate. For instance, the substituents of a substituted alkyl may include substituted and unsubstituted forms of aminos, azidos, iminos, amidos, phosphoryls (including phosphonates and phosphinates), sulfonyls (including sulfates, sulfonamidos, sulfamoyls and sulfonates), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “aryl” as used herein refers to a C₆₋₁₂ monocyclic or bicyclic hydrocarbon ring wherein at least one ring is aromatic. Examples of such groups include phenyl, naphthyl or tetrahydronaphthalenyl and the like.

The term “heteroaryl” as used herein refers to a 5-6 membered monocyclic aromatic or a fused 8-10 membered bicyclic aromatic ring containing 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such fused aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and the like.

As mentioned above, the invention also includes pharmaceutically acceptable salts of the above-mentioned compounds (e.g., compounds of formula I or Ia). A compound of the invention can possess a sufficiently acidic functionality, a sufficiently basic functionality, or both functional groups. Accordingly, a compound may react with any of a number of inorganic bases, and organic and inorganic acids, to form a pharmaceutically acceptable salt.

The term “pharmaceutically acceptable salt” as used herein refers to salts of the compounds of formula I or Ia which are substantially non-toxic to living organisms. Typical pharmaceutically acceptable salts include those salts prepared by reaction of the compounds of the present invention with a pharmaceutically acceptable mineral or organic acid or an inorganic base. Such salts are known as acid addition and base addition salts.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogen phosphate, dihydrogen phosphate, metaphosphate, pyrophosphate, chloride, bromide, iodide, acetate, propionate, decanoate, caprylate, acrylate, formate, isobutyrate, caproate, heptanoate, propionate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylene-sulfonate, phenylacetate, phenyipropionate, phenylbutyrate, citrate, lactate, γ-hydroxybutyrate, glycolate, tartrate, methanesulfonate, propanesulfonate, naphthalene-1-sulfonate, napthalene-2-sulfonate, mandelate and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. Suitable organic bases include trialkylamines such as triethylamine, procaine, dibenzylamine, N-benzyl-β-phenethyl-amine, 1-ephenamine, N,N′-dibenzylethylene-diamine, dehydroabietylamine, N-ethylpiperidine, benzylamine, dicyclohexylamine, or the like pharmaceutically acceptable amines. In an embodiment, the above-mentioned salt is a potassium salt or a sodium salt.

In another aspect, the present invention provides a composition comprising the above-mentioned compound and a pharmaceutically acceptable carrier or excipient. The compounds (e.g., the compounds of formula I and/or Ia) may be administered in the form of pharmaceutical compositions. They can be administered by a variety of routes including oral, rectal, transdermal, subcutaneous, intravenous, intramuscular, intranasal and intrathecal. The compounds are effective as both injectable and oral compositions. Such compositions are prepared in a manner well known in the pharmaceutical art and comprise at least one active compound and a pharmaceutically acceptable diluent or carrier or excipient. Supplementary active compounds can also be incorporated into the compositions.

In making the compositions employed in the present invention, the active ingredient (e.g., a compound of formula I and/or Ia) is usually mixed with a pharmaceutically acceptable carrier or excipient. As used herein “pharmaceutically acceptable carrier” or “excipient” includes any and all solvents, buffers, dispersion media, coatings, antibacterial and antifungal agents, isotonic and absorption delaying agents, and the like that are physiologically compatible. The carrier can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration (see Remington: The Science and Practice of Pharmacy by Alfonso R. Gennaro, 2003, 21^{th} edition, Mack Publishing Company).

Formulations suitable for oral administration can consist of (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds/compositions of the invention include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

For preparing pharmaceutical compositions from the compound(s)/composition(s) of the present invention, pharmaceutically acceptable carriers are either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired. The powders and tablets may typically contain from 5% or 10% to 70% of the active compound/composition. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use are prepared by dissolving the active compound(s)/composition(s) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

In an embodiment, a compound of the invention (e.g., a compound of formula I or Ia, or a pharmaceutically-acceptable salt thereof) is administered such that it comes into contact with neural cells or neural tissue, such as central nervous system (CNS) cells or tissue. Such tissue includes brain and spinal cord (e.g., cervical, thoracic, or lumbar) tissue. As such, in embodiments a compound of the invention can be administered to treat neural cells/tissue in vivo via direct intracranial injection or injection into the cerebrospinal fluid. Alternatively, the compound can be administered systemically (e.g. intravenously) and may come into contact with the affected neural tissue via lesions (where the blood-brain barrier is compromised), or, in a further embodiment, may be in a form capable of crossing the blood-brain barrier and entering the neural system (e.g., CNS). Further, in an embodiment, a composition of the invention may be formulated for such administration to neural cells/tissue.

Formulations to be used for in vivo administration are preferably sterile. This is readily accomplished, for example, by filtration through sterile filtration membranes.

The composition may also contain more than one active compound for the particular indication being treated, preferably those with complementary activities that do not adversely affect each other. It may be desirable to use the above-mentioned composition in addition to one or more agents currently used to prevent or treat the disorder in question. The above-mentioned agents may be formulated in a single composition or in several individual compositions which may be co-administered in the course of the treatment.

The amount of the pharmaceutical composition (e.g., a compound of formula I or Ia, or a salt thereof) which is effective in the prevention and/or treatment of a particular disease, disorder or condition (e.g., inflammatory disease, neural injury) will depend on the nature and severity of the disease, the chosen prophylactic/therapeutic regimen, the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present invention should be sufficient to effect a beneficial prophylactic and/or therapeutic response in the patient over time. The size of the dose also will be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

In another aspect, the present invention provides a method for inhibiting PLA₂ activity in a system, e.g., a cell, cell-free system, biological system, or a subject, said method comprising contacting said system with, or administering to said subject, an effective amount of the above-mentioned compound or composition.

In another aspect, the invention provides a method for preventing and/or treating an inflammatory disease or condition in a subject, said method comprising administering to said subject an effective amount of the above-mentioned compound or composition.

In another aspect, the present invention provides the use of the above-mentioned compound or composition for the prevention and/or treatment of an inflammatory disease or condition.

In yet another aspect, the present invention provides the use of the above-mentioned compound or composition for the preparation of a medicament for the prevention and/or treatment of a neural disease or condition.

An “effective amount” refers to an amount effective, at dosages and for periods of time necessary, to achieve the desired prophylactic or therapeutic result. An effective amount refers to the amount of active compound or pharmaceutical agent that elicits the biological or medicinal response in a tissue, system, animal, individual or human that is being sought by a researcher, veterinarian, medical doctor or other clinician, which includes one or more of the following:

(A) Preventing the disease; for example, preventing a neural disease, condition or disorder and/or an inflammatory disease, condition or disorder in an individual that may be predisposed to the disease, condition or disorder but does not yet experience or display the pathology or symptomatology of the disease,

(B) Inhibiting the disease; for example, inhibiting a neural disease, condition or disorder and/or an inflammatory disease, condition or disorder in an individual that is experiencing or displaying the pathology or symptomatology of the disease, condition or disorder (i.e., arresting further development of the pathology and/or symptomatology), and

(C) Ameliorating the disease; for example, ameliorating neural disease, condition or disorder and/or an inflammatory disease, condition or disorder in an individual that is experiencing or displaying the pathology or symptomatology of the disease, condition or disorder (i.e., reversing the pathology and/or symptomatology).

An effective amount of a compound or composition of the present invention may vary according to factors such as the disease state, age, sex, and weight of the individual, and the ability of the compound to elicit a desired response in the individual. Dosage regimens may be adjusted to provide the optimum prophylactic or therapeutic response. An effective amount is also one in which any toxic or detrimental effects of the compound are outweighed by the therapeutically beneficial effects. It will be understood that the amount of the compound actually administered will be determined by a physician, in the light of the relevant circumstances, including the condition to be treated, the chosen route of administration, the actual compound administered, the age, weight, and response of the individual patient, and the severity of the patient's symptoms, and therefore the above dosage ranges are not intended to limit the scope of the invention in any way. In some instances dosage levels below the lower limit of the aforesaid range may be more than adequate, while in other cases still larger doses may be employed without causing any harmful side effect, provided that such larger doses are first divided into several smaller doses for administration throughout the day.

In embodiments, the above-mentioned treatment may be effected prior to, after, or both prior to and after the onset of symptom(s) of a neural disease or condition. For example, a compound or composition of the invention (e.g., a compound of formula I or Ia, or a salt thereof, or a composition comprising a compound of formula I or Ia, or a salt thereof and a pharmaceutically-acceptable carrier) may be administered to a subject prior to, after, or both prior to and after the onset of symptom(s) of a neural disease or condition. Similarly, the invention provides a use of a compound or composition of the invention (e.g., a compound of formula I or Ia, or a salt thereof, or a composition comprising a compound of formula I or Ia, or a salt thereof and a pharmaceutically-acceptable carrier) for the treatment of, or for the preparation of a medicament for the treatment of, a neural disease or condition, wherein the use is prior to, after, or both prior to and after the onset of symptom(s) of the neural disease or condition.

Neural disease or disorder or condition as used herein includes, for example, traumatic brain injury, spinal cord injury (SCI), fronto-temporal dementias (tauopathies), peripheral neuropathy, Parkinson's disease, Huntington's disease, multiple sclerosis (MS), Alzheimer's disease and amyotropic lateral sclerosis (ALS). In an embodiment, the above-mentioned disease or condition is a neural injury. In a further embodiment, the above-mentioned neural injury is spinal cord injury. In embodiments, the above-mentioned treatment results in one or more of: increased locomotion and control (e.g., increased BMS subscores), increased tissue sparing (i.e. decreased tissue damage), increased myelin sparing (i.e. decreased myelin damage), increased sparing and/or regeneration of serotonergic fibers.

In yet another embodiment, the above-mentioned neural disease or condition is a demyelinating disease. In a further embodiment, the above-mentioned demyelinating disease is Multiple Sclerosis (MS).

In an embodiment, the above-mentioned disease/condition/disorder is associated with inflammation (e.g., atherosclerosis). In a further embodiment, the above-mentioned disease/condition/disorder is an inflammatory disease or condition of the central nervous system (CNS). In a further embodiment, the above-mentioned neural disease/condition/disorder or inflammation is associated with PLA₂ activity. In a further embodiment, the above-mentioned PLA₂ activity is iPLA₂ activity. In a further embodiment, the above-mentioned iPLA₂ activity is group VIA iPLA₂ activity.

Phospholipase A₂ (PLA₂) consists of a family of phospholipid enzymes that play a normal physiological role in phospholipid metabolism, inflammation, host defense, and signal transduction (Brown W. J. et al., (2003) Traffic 4(4): 214-21). They hydrolyze an ester bond at sn-2 position of phospholipids that generates a free fatty acid such as arachidonic acid (AA) and a lysophospholipid such as lysophosphatidylcholine (LPC) (Murakami et al., (1997) Crit. Rev. Immunol. 17, 225-83; Dennis, E. A. (1994). J. Biol. Chem. 269: 13057-60). Arachidonic acid can give rise to eicosanoids via cyclooxygenase (COX-1 and 2) and 5-lipoxygenase (5-LO) enzymes. Eicosanoids such as prostaglandins, thromboxanes, and leukotrienes are potent mediators of inflammation by increasing vascular permeability and inducing chemotaxis of immune cells (Dennis, E. A. et al. (1991). FASEB J. 5: 2068-77). In addition, LPC is a myelinolytic agent and can act as a chemoattractant for immune cells (Ousman, S. S. and David, S. (2000). Glia. 30: 92-104; Ryborg, A. K. et al. (1994). Arch Dermatol Res. 286: 462-5; Ryborg, A. K. et al. (2000). Acta. Derm. Venereol. 80: 242-6). Injection of LPC into the spinal cord causes demyelination as well as inducing the expression of a number of chemokines and cytokines (Ousman et al., 2000, supra; Ousman, S. S. and David, S. (2001). J. Neurosci. 21: 4649-56). An abnormally high expression of PLA₂ in the CNS could therefore lead to inflammation and demyelination.

PLA₂ enzymes fall broadly into three main groups: the group IV cytosolic PLA₂ (cPLA₂), the group II secretory PLA₂ (sPLA₂), and the group VI Ca²⁺-independent PLA₂ (referred to as iPLA₂) (Murakami et al. (2002). J. Biochem. 131: 285-292). The secreted form is a low molecular weight form (14 kDa) that has no preference for the type of fatty acid at the sn-2 position of phospholipids (Murakami et al. (1997), supra; Dennis et al. (1994), supra). Members of the cytosolic form have a higher molecular mass (85-110 kDa) and selectively hydrolyze phospholipids containing AA. iPLA₂s are divided into two groups, VIA and VIB, and are generally regarded as housekeeping enzymes for the maintenance/remodeling of membrane phospholipids (Kudo I. and Murakami M. (2002). Prostaglandins Other Lipid Mediat. 68-69: 3-58).

As noted above, PLA₂ (EC 3.1.1.4, CAS Registry Number: 9001-84-7) catalyzes the hydrolysis of the sn-2 position of a glycerophospholipid to liberate fatty acid and a lysophospholipid lacking the fatty acid at the 2 position of the glycerol backbone. For example, PLA₂ can act on membrane phospholipids to release arachidonic acid (AA), a precursor of eicosanoids including prostaglandins (PGs) and leukotrienes (LTs) (Murakami et al. (2002). J. Biochem. 131: 285-292). As noted above, the same reaction also produces lysophosholipids, which represent another class of lipid mediators. So far, at least 19 enzymes that possess PLA₂ activity have been identified in mammals. The secretory PLA₂ (sPLA₂) family, in which 10 isozymes have been identified, consists of low-molecular weight, Ca²⁺-dependent, secretory enzymes that have been implicated in a number of biological processes including modification of eicosanoid generation, inflammation, host defense, and atherosclerosis. The cytosolic PLA₂, (cPLA₂) family consists of 3 enzymes, among which cPLA₂α plays an essential role in the initiation of AA metabolism. Intracellular activation of cPLA₂α is tightly regulated by Ca²⁺ and phosphorylation. The Ca²⁺-independent PLA₂ (iPLA₂) family contains 2 enzymes and may play a major role in membrane phospholipid remodeling. The structure of PLA₂s is described in Murakami et al. (2002). J. Biochem. 131: 285-292.

The following GenBank accession numbers represent examples of nucleic acid sequences encoding several isoforms of enzymes having PLA₂ activity: NM_{—}001004426, NM_{—}003560, NM_{—}024420, NM_{—}178034, NM_{—}005090, NM_{—}213600, NM_{—}003706, NM_{—}000928, NM_{—}005084, NM_{—}001080490, NM_{—}012400, NM_{—}003561, NM_{—}000929, NM_{—}022819, NM_{—}022819, NM_{—}032562, NM_{—}030821, NM_{—}000300 and NM_{—}014589.

The following GenBank accession numbers represent examples of amino acid sequences of several isoforms of enzymes having PLA₂ activity: NP_{—}077734, NP_{—}000291, NP_{—}001004426, NP_{—}003551, NP_{—}000919, NP_{—}003697, NP_{—}000920, NP_{—}036532, NP_{—}003552, NP_{—}005081, NP_{—}005075, NP_{—}110448, NP_{—}828848, NP_{—}073730, NP_{—}115951, NP_{—}998765, NP_{—}056530, NP_{—}055404, NP_{—}001073959 and NP_{—}056538.

As used herein, the term “inhibition” refers to a decrease in activity, and in the context of PLA₂ activity, refers to a decrease in measurable PLA₂ activity (e.g., enzymatic activity), in an embodiment by at least 10% relative to a reference or control (e.g., in a corresponding PLA₂-containing sample that has not been contacted with or subjected to the inhibitor/conditions in respect of which inhibition is being assessed). In further embodiments, such inhibition is at least 20%, 30%, 40%, 50%, 60%, 70%, 80%, 90% or more, up to and including 100%, i.e. complete inhibition or absence of the given PLA₂ activity. Methods for measuring PLA₂ activity are well known in the art (see, for example, Kokotos, G. et al. (2002) J. Med. Chem. 45: 2891-2893; Kokotos, G. et al. (2004). J. Med. Chem. 47: 3615-3628; Stephens D. et al. (2006). J. Med. Chem. 49: 2821-2828).

In an embodiment, the above-mentioned PLA₂ activity is cPLA₂ or iPLA₂ activity. In another embodiment, the above-mentioned use or method inhibits sPLA₂ activity to a lesser extent than it inhibits cPLA₂ or iPLA₂ activity. In a further embodiment, the above-mentioned use or method does not significantly inhibit sPLA₂ activity.

In an embodiment, the above-mentioned prevention/treatment comprises the use/administration of more than one (i.e. a combination of) active agent (e.g., one or more compounds of formula I or Ia, or salts thereof). The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) of the present invention is used/administered in combination with one or more agent(s) currently used to prevent or treat the disorder in question.

The invention further provides kits or packages (e.g., commercial kits or packages) comprising the above-mentioned compositions or agents together with instructions for their use for the prevention or treatment of a neural disease or condition in a subject (e.g., an inflammatory disease or condition of the central nervous system, or a neural injury). The kit or package may further comprise other components, such as buffers, containers and/or devices for administering the agent/composition to a subject.

As used herein, the terms “subject” or “patient” are used interchangeably are used to mean any animal, such as a mammal, including humans and non-human primates. In an embodiment, the above-mentioned subject is a mammal. In a further embodiment, the above-mentioned subject is a human.

The articles “a,” “an” and “the” are used herein to refer to one or to more than one (i.e., to at least one) of the grammatical object of the article.

The present invention is illustrated in further details by the following non-limiting examples.

### Examples

### Example 1

### Materials and Methods

EAE mice: EAE was induced in female SJL/J mice by subcutaneous injections of 100 μg of proteolipid protein (PLP) (Sheldon Biotechnology Centre, Montreal, Canada) in Complete Freund's Adjuvant (CFA) [Incomplete Freund's adjuvant containing 4 mg/ml of heat inactivated Mycobacterium tuberculosis (Fisher Scientific, Nepean, Canada)]. They were then boosted on day 7 with 50 μg of PLP in CFA containing 2 mg/ml of heat inactivated Mycobacterium tuberculosis. The mice were monitored daily for clinical symptoms of EAE using the following 5-point scale: Grade 0=normal (no clinical signs), Grade 1=flaccid tail, Grade 2=mild hindlimb weakness (fast righting reflex), Grade 3=severe hindlimb weakness (slow righting reflex), Grade 4=hindlimb paralysis, Grade 5=hindlimb paralysis and forelimb weakness or moribund. The clinical monitoring was done in a blind fashion.

Detection of PLA₂ by RT-PCR (EAE experiments, Example 15). Spinal cords and spleens were removed from animals at the onset, peak, and remission stages of disease, and RNA isolated using the RiboPure™ kit (Ambion Inc, Austin, Tex.) and reverse transcribed to cDNA. RT-PCR was performed using the GeneAmp™ RNA PCR kit (PerkinElmer Life Sciences). Primers used were as follows:

| | |
|---|---|
| cPLA₂ IVA: | |
| U-5′-ATGCCGCCCGCCTGTCCTT-3′; | (SEQ ID NO: 1) |
| L-5′-GGGTCCTTGAGCCTCATCATCA-3′; | (SEQ ID NO: 2) |
| iPLA₂ VIA, | |
| U-5′-GGTGCGCGTCCTGCTGCTCTGTA-3′; | (SEQ ID NO: 3) |
| L-5′AGTGGCGTGTTCCCGTGCTCTCC-3′. | (SEQ ID NO: 4) |

PCR was performed as described previously (Jeong, S. Y. and David, S. (2003). J. Biol. Chem. 278: 27144-8) with annealing temperatures of 57° C. (for cPLA₂ IVA) and 60° C. (for iPLA₂ VIA and GAPDH).

Detection of PLA₂ by RT-PCR (spinal cord injury experiments, Example 19). RNA from 5 mm length of spinal cord tissue containing the lesion site harvested at 1, 3, 7, 14, 21 and 28 days post-lesion was extracted using RNeasy™ Lipid Tissue kit (Qiagen, Mississauga, Ontario, Canada). PCR amplification was performed with specific primers for mammalian PLA₂s family members as previously described (Kalyvas, A. and David, S. (2004). Neuron 41: 323-35). Peptidylprolyl isomerase A (PPIA) was used as controls to ensure equal cDNA samples for PCR amplification. Six spinal cords were pooled for each time point.

Double Immunofluorescence (EAE experiments). Mice at different clinical stages of EAE (onset, peak, remission) were deeply anesthetized with ketamine:xylazine:acepromazine (50:5:1 mg/kg) and perfused with 0.1 M phosphate buffer followed by perfusion with 4% paraformaldehyde in 0.1 M phosphate buffer. Cryostat sections (12 μm) were blocked in 0.1% Triton™-X 100 and 10% normal goat serum and incubated overnight with anti-cPLA₂ (Santa Cruz Biotechnology, Santa Cruz, Calif., 1:75) or anti-iPLA₂ (Cayman Chemical, 1:500) combined with a monoclonal antibodies specific for astrocytes (rat anti-GFAP, Sigma, 1:1000). This was followed by incubation with a biotinylated goat anti rabbit secondary antibody (Jackson ImmunoResearch Laboratories, West Grove, Pa., 1:400) combined with a goat anti-rat rhodamine-conjugated secondary antibody (Jackson ImmunoResearch Laboratories, West Grove, Pa., 1:200). After washing, the sections were incubated with fluorescein-conjugated streptavidin (Molecular Probes, Eugene, Oreg., 1:400).

Flow cytometry. The CNS was removed from 6 animals at each clinical stage: onset peak and remission. A single cell suspension was made and the immune cells were isolated using a Percoll™ gradient. The cells were then stained with an anti-cPLA₂ or iPLA₂ antibody, in combination with one of the immune cell type specific antibodies: anti-CD4-FITC, anti-CD8-FITC, anti-CD11b-FITC or anti-CD11c-FITC antibodies (BD Pharmingen, 1:200). This was followed by incubation with a biotinylated goat anti-rabbit secondary antibody, and then a PE-conjugated streptavidin. Data was collected on a FACScan™ or a FACSCalibur™ and analyzed using CellQuest™ Pro (BD Biosciences).

Treatment of EAE-induced mice. EAE-induced mice were randomly assigned to each of the treatment and control groups. For the groups that received treatment before the onset of clinical symptoms, treatment was started on day 6 after immunization and given daily for 3 weeks. Daily injections of the fluoroketone compounds (FKGK11 and FKGK2) were given on a 3-day cycle consisting of one intravenous injection followed by 2 intraperitoneal injections. Mice in the control group were treated with PBS containing 5% Tween™ 80. For the groups that received delayed treatment after symptoms occurred, mice were treated with daily intraperitoneal injections of FKGK11 and FKGK2 starting from the first day of clinical symptoms, beginning on day 11 for 2 weeks.

Mouse Inflammation antibody array. Spinal cords were removed from vehicle- and FKGK11-treated animals at the peak stage of disease, and were then homogenized and centrifuged at 1000×g. The proteins were extracted and analyzed using the RayBio® Mouse Inflammation Antibody Array 1.1 (Cat. #0106008/AAM-INF-1; RayBiotech Inc.; Norcross, Ga.), which simultaneously detects 40 cytokines and other related proteins. Densitometry analysis was performed to detect differences between the various inflammatory mediators.

Spinal cord contusion and drug administration. All surgical procedures were approved by the McGill University Animal Care Committee and followed the guidelines of the Canadian Council on Animal Care. Adult (8-10 weeks old) female BALB/c (Charles River Canada), cPLA₂ IVA^{-/-} and wild type littermates mice, were anesthetized with ketamine:xylazine:acepromazine (50:5:1 mg/kg). After performing a laminectomy at the 11^{th} thoracic vertebrae, the exposed spinal cord was contused using the Infinite Horizons Impactor™ device (Precision Scientific Instrumentation, Lexington, Ky.). Moderate injuries were made using a force of 50 kDynes, and only animals that had tissue displacement ranged between 400-600 μm were used (Ghasemblou et al. (2005). Exp Neurol. 196(1): 9-17).

PLA₂ inhibitor treatment. Mice were given daily intraperitoneal injections of 2 mM fluoroketone (iPLA₂ inhibitor; FKGK11) in 200 μl (6.85 mg/kg), starting 1 h after contusion and for 14 days. The control group that also had SCI were treated daily with vehicle.

Western blotting. Protein was extracted from 5 mm length of spinal cord tissue containing the lesion site harvested at the same time points that were used for the RT-PCR experiments (1, 3, 7, 14, 21 and 28 days post-lesion). Protein samples (20 μg) were separated on a 4-12% Bis-Tris gel (Invitrogen) and transferred onto PVDF membranes (Millipore). The membranes were incubated with antibodies against iPLA₂ VI (Cayman Chemical) and bands were detected using Chemiluminescence™ (Western Lightning Chemiluminescence Reagent Plus, PerkinElmer). β-actin (Sigma Aldrich) was used to ensure equal loading of samples. Three samples were used for each time point.

Functional assessment. Locomotor recovery was evaluated in an open-field test using the Basso Mouse Scale (BMS) (Basso D. M. et al., (2006). J Neurotrauma. 23(5): 635-59). The BMS analysis of hindlimb movements and coordination was carried out by two independent assessors and the consensus score taken. The final score is presented as mean±SEM.

Histology. Mice were perfused with 4% paraformaldehyde in 0.1M phosphate buffer (PB) at 1, 3, 7, 14 and 28 days post-lesion. 5 mm length of the spinal cord containing the lesion site was removed, cryoprotected with 30% sucrose in 0.1M PB, and cut in serial sections (16 μm thick). For double immunoflorescence, sections were incubated with antibodies against iPLA₂ VIA (Cayman Chemical) combined with antibodies against Mac-1 (for macrophages/microglia, Serotec), GFAP (for astrocytes, Zymed Labs), CC1 (for oligodendrocytes, Calbiochem), NeuN (for neurons, 1:500, Chemicon) and SMI312 (for axons, Covance). Immunfluorescence labeling for 5-hydroxytryptamine (5-HT) (Sigma, Aldreich) was also performed to assess innervation of serotonergic axons caudal to the lesion. In addition, one series of serial sections of the spinal cord were stained with Luxol fast blue (LFB) histochemistry, which stains myelin, and another series stained with cresyl violet histochemistry to quantify neuronal loss.

Quantification of histological results. Histological quantification was performed from spinal cord sections harvested at 28 days post-lesion. Tissue sections were viewed with an Axioskop™ 2 Plus microscope (Zeiss) and images captured using a Qlmaging Retiga™ 1300 camera, and quantification done using BioQuant™ Nova Prime image analysis system (BioQuant Image Analysis Corp.). Tissue and myelin sparing was calculated by delineating the GFAP and LFB stained sections. Neuronal survival was assessed by counting the neuron profiles in the ventral horn of the spinal cord in tissue sections stained with cresyl violet. Assessment of serotonergic innervation was performed by calculating the area occupied by serotoninergic axons in the lateral funiculi and ventral horns of spinal cord sections taken at a distance of 1000 μm caudal to the lesion site.

Statistical analyses (EAE experiments, Example 15-18). Data are shown as mean±s.e.m. RT-PCR analyses were done using the student's T test. Statistical analyses in the functional assessments were performed by using two way repeated measures Friedman's ANOVA on Ranks. Differences were considered significant if p<0.05.

Statistical analyses (spinal cord injury experiments, Examples 19-20). Data are shown as mean±s.e.m. RT-PCR and Western blot analyses were done using one-way ANOVA with post-hoc Dunnett's test. Statistical analyses of the functional and histological assessments were performed using two-way repeated measures ANOVA with post-hoc Tukey's test for multiple comparisons. Differences were considered significant at p<0.05.

Inhibition of phospholipase A₂ by perfluoroketone compounds. The compounds were tested for their ability to inhibit human GIVA cPLA₂ in a GIVA cPLA₂ specific assay that uses mixed micelles of substrate, 1-palmitoyl-2-arachidonyl phosphatidylcholine, phosphatidylinositol 4,5-bisphosphate and detergent Triton™ X-100 (97:3:400 μM), as previously described (Kokotos, G. et al. (2002) J. Med. Chem. 45: 2891-2893; Kokotos, G. et al. (2004). J. Med. Chem. 47: 3615-3628). The standard GVIA iPLA₂ activity assay utilizes DPPC/Triton™ X-100 mixed micelles at a ratio of 1:4 as previously described (Stephens D. et al. (2006). J. Med. Chem., 49: 2821-2828). GV sPLA₂ activity was measured in an assay similar to the assays for GIVA cPLA₂ and GVIA iPLA₂. Briefly, the reaction monitored the release of [¹⁴C]-palmitic acid from phospholipid-detergent mixed micelles containing 1-palmitoyl-2-[¹⁴C]-palmitoyl phosphatidylcholine (DPPC) and Triton™ X-100 (1:4 ratio).

### Example 2

### Synthesis and Characterization of Pentafluoroethyl Ketones

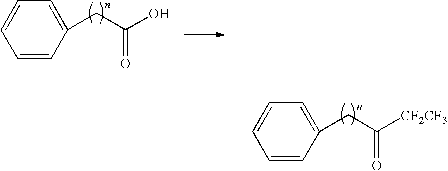

Oxalyl chloride (0.38 g, 3 mmol) and N,N-dimethylformamide (40 μL) were added to a solution of carboxylic acid (1 mmol) in dry dichloromethane (40 mL). After 3 h stirring at room temperature, the solvent and excess reagent were evaporated under reduced pressure and the residue was dissolved in dry dichloromethane (10 mL). Pyridine (0.64 mL, 8 mmol) and pentafluoropropionic anhydride (0.85 mL, 6 mmol) were added dropwise to this solution at 0° C. consecutively. After stirring at 0° C. for 30 min and at room temperature for 1.5 h, the reaction mixture was cooled again at 0° C. and water (2 mL) was added dropwise. After stirring for 30 min at 0° C. and another 30 min at room temperature, the reaction mixture was diluted with dichloromethane (10 ml). The organic phase was then washed with brine and dried (Na₂SO₄). The solvent was evaporated under reduced pressure and the residual oil was purified by column chromatography (ethyl acetate/petroleum ether 1/9).

### 1,1,1,2,2-Pentafluoro-7-phenyl-3-heptanone (FKGK11)

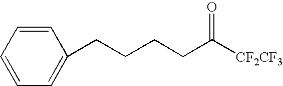

Yellowish oil, yield=53%; ¹H NMR (CDCl₃): δ 7.19 (m, 5H, Ph), 2.80 (t, J=6.2 Hz, 2H, CH₂), 2.70 (t, J=6.6 Hz, 2H, CH₂), 1.69 (m, 4H, 2×CH₂); ¹³C NMR (CDCl₃): δ 194.2 (t, J_{CCF2}=26 Hz, CO), 141.6 (Ph), 128.4 (Ph), 128.3 (Ph), 125.9 (Ph), 117.8 (qt, J_{CF3}=287 Hz, J_{CCF2}=34 Hz, CF₃), 106.8 (tq, J_{CF2}=267 Hz, J_{CCF3}=38 Hz, CF₂), 37.1 (CH₂), 35.5 (CH₂), 30.3 (CH₂), 21.9 (CH₂); ¹⁹F NMR (CDCl₃): δ −4.1 (CF₃), −45.5 (CF₃); MS (ESI) m/z (%): 279 (M⁻,100).

### 1,1,1,2,2-Pentafluoro-8-phenyl-3-octanone (FKGK12)

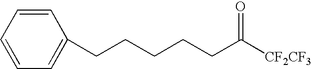

Yellowish oil, yield=95%; ¹H NMR (CDCl₃): δ 7.25 (m, 5H, Ph), 2.79 (t, J=6.8 Hz, 2H, CH₂), 2.68 (t, J=7.4 Hz, 2H, CH₂), 1.75 (m, 4H, 2×CH₂), 1.44 (m, 2H, CH₂); ¹³C NMR (CDCl₃): δ 194.3 (t, J_{CCF}=26 Hz, CO), 142.2 (Ph), 128.3 (Ph), 128.3 (Ph), 125.7 (Ph), 117.8 (qt, J_{CF3}=285 Hz, J_{CCF2}=34 Hz, CF₃), 106.9 (tq, J_{CF2}=265 Hz, J_{CCF3}=37 Hz, CF₂), 37.2 (CH₂), 35.6 (CH₂), 31.0 (CH₂), 28.2 (CH₂), 22.1 (CH₂); ¹⁹F NMR (CDCl₃): δ −4.1 (CF₃), −45.5 (CF₂); MS (ESI) m/z (%): 293 (M⁻,100).

### 1,1,1,2,2-Pentafluoro-9-phenyl-3-nonanone (FKGK13)

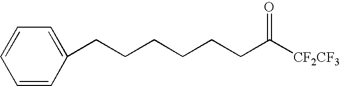

Yellowish oil, yield =60%; ¹H NMR (CDCl₃): δ 7.25 (m, 5H, Ph), 2.76 (t, J=6.8 Hz, 2H, CH₂), 2.64 (t, J=8.0 Hz, 2H, CH₂), 1.67 (m, 4H, 2×CH₂), 1.38 (m, 4H, 2×CH₂); ¹³C NMR (CDCl₃): δ 194.4 (t, J_{CCF}=26 Hz, CO), 142.4 (Ph), 128.4(Ph), 128.3 (Ph), 125.7 (Ph), 117.8 (qt, J_{CF3}=285 Hz, J_{CCF2}=34 Hz, CF₃), 106.9 (tq, J_{CF2}=265 Hz, J_{CCF3}=37 Hz, CF₂), 37.3 (CH₂), 35.8 (CH₂), 31.1 (CH₂), 28.8 (CH₂), 28.5 (CH₂), 22.2 (CH₂); ¹⁹F NMR (CDCl₃): δ −4.1 (CF₃), −45.6 (CF₂); MS (ESI) m/z (%): 307 (M⁻,100).

### 1,1,1,2,2-Pentafluoro-octadecan-3-one

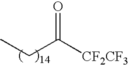

Colorless oil, yield=24%; ¹H NMR (CDCl₃): δ 2.75 (t, J=7.4 Hz, 2H, CH₂), 1.67 (t, J=7 Hz, 2H, CH₂), 1.27 (m, 22H, 11×CH₂), 0.88 (t, J=7 Hz, 3H, CH₃); ¹³C NMR (CDCl₃): δ 194.5 (t, J_{CCF2}=26 Hz, CO), 117.8 ppm (qt, J_{CF3}=285 Hz, J_{CCF2}=34 Hz, CF₃), 106.9 ppm (tq, J_{CF2}=265 Hz, J_{CCF3}=38 Hz, CF₂), 37.3 (CH₂), 31.9 (CH₂), 30.3 (CH₂), 29.7 (CH₂), 29.6 (CH₂), 29.5 (CH₂), 29.4 (CH₂), 29.2 (CH₂), 28.7 (CH₂), 22.7 (CH₂), 22.3 (CH₂), 14.1 (CH₃); ¹⁹F NMR (CDCl₃, TFA): δ −4.1 (CF₃), −45.5 (CF₂); MS (ESI) m/z (%): 357 (M⁻,100).

### 1,1,1,2,2-Pentafluoro-7-naphthalen-2-yl-heptan-3-one (FKGK17)

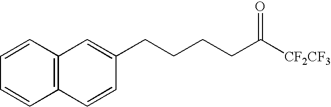

Yellowish oil, yield=38%; ¹H NMR (CDCl₃): δ 7.85 (m, 3H, Ph), 7.65 (s, 1H, Ph), 7.51-7.23 (m, 3H, Ph), 2.81 (m, 4H, 2×CH₂), 1.78 (m, 4H, 2×CH₂); ¹³C NMR (CDCl₃): δ 194.4 (t, J_{CCF}=26 Hz, CO), 139.4 (Ph), 133.9 (Ph), 132.3 (Ph), 127.6 (Ph), 127.4 (Ph), 127.2 (Ph), 126.4 (Ph), 125.9 (Ph), 125.4 (Ph), 117.8 (qt, J_{CF3}=285 Hz, J_{CCF2}=34 Hz, CF₃), 106.9 (tq, J_{CF2}=265 Hz, J_{CCF3}=37 Hz, CF₂), 37.4 (CH₂), 35.9 (CH₂), 30.5 (CH₂), 22.2 (CH₂); ¹⁹F NMR (CDCl₃): δ −4.1 (CF₃), −45.5 (CF₂); MS (ESI) m/z (%): 329 (M⁻,100).

Following a similar procedure, the corresponding trifluoromethyl ketone was prepared.

### 1,1,1-Trifluoro-6-naphthalen-2-yl-hexan-2-one (FKGK18)

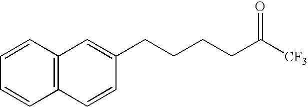

Yellowish oil, yield=39%; ¹H NMR (CDCl₃): δ 7.81 (m, 3H, Ph), 7.61 (s, 1H, Ph), 7.48-7.25 (m, 3H, Ph), 2.78 (m, 4H, 2×CH₂), 1.75 (m, 4H, 2×CH₂); ¹³C NMR (CDCl₃): δ 191.6 (t, J_{CCF}=26 Hz, CO), 139.4 (Ph), 133.9 (Ph), 132.3 (Ph), 127.6 (Ph), 127.4 (Ph), 127.1 (Ph), 126.4 (Ph), 125.9 (Ph), 125.2 (Ph), 115.8 (q, J_{CF}=292 Hz, CF₃), 36.2 (CH₂), 35.6 (CH₂), 30.3 (CH₂), 22.0 (CH₂); ¹⁹F NMR (CDCl₃): δ −1.5 (CF₃).

### Example 3

### Synthesis and Characterization of Heptafluoropropyl Ketones

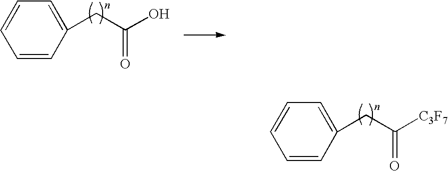

Oxalyl chloride (0.38 g, 3 mmol) and N,N-dimethylformamide (40 μL) were added to a solution of carboxylic acid (1 mmol) in dry dichloromethane (40 mL). After 3 h stirring at room temperature, the solvent and excess reagent were evaporated under reduced pressure and the residue was dissolved in dry dichloromethane (10 mL). Pyridine (0.64 mL, 8 mmol) and heptafluorobutanoic anhydride (1.46 mL, 6 mmol) were added dropwise to this solution at 0° C. consecutively. After stirring at 0° C. for 30 min and at room temperature for 1.5 h, the reaction mixture was cooled again at 0° C. and water (2 mL) was added dropwise. After stirring for 30 min at 0° C. and another 30 min at room temperature, the reaction mixture was diluted with dichloromethane (10 mL). The organic phase was then washed with brine and dried (Na₂SO₄). The solvent was evaporated under reduced pressure and the residual oil was purified by column chromatography (ethyl acetate/petroleum ether 5/95).

### 1,1,1,2,2,3,3-Heptafluoro-8-phenyl-octan-4-one (FKGK19)

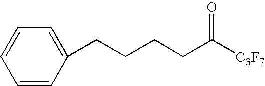

Yellowish oil, yield 59%; ¹H NMR (CDCl₃): δ 7.22 (m, 5H, Ph), 2.76 (t, J=6.2 Hz, 2H, CH₂), 2.65 (t, J=6.6 Hz, 2H, CH₂), 1.69 (m, 4H, 2×CH₂); ¹³C NMR (CDCl₃); δ 193.9 (t, J_{CCF2}=26 Hz, CO), 141.6 (Ph), 128.4 (Ph), 128.3 (Ph), 126.6-102.0 (m, 2×CF₂, CF₃), 125.9 (Ph), 37.8 (CH₂), 35.5 (CH₂), 30.3 (CH₂), 21.9 (CH₂); ¹⁹F NMR (CDCl₃): δ −9.3 (CF₃), −49.9 (CF₂), 55.4 (CF₂).

### 1,1,1,2,2,3,3-Heptafluoro-9-phenyl-nonan-4-one (FKGK20)

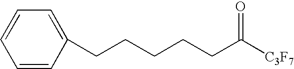

Yellowish oil, yield 76%; ¹H NMR (CDCl₃): δ 7.23 (m, 5H, Ph), 2.74 (t, J=6.2 Hz, 2H, CH₂), 2.63 (t, J=6.6 Hz, 2H, CH₂), 1.69 (m, 4H, 2×CH₂), 1.38 (m, 2H, CH₂); ¹³C NMR (CDCl₃): δ 194.4 (t, J_{CCF2}=26 Hz, CO), 142.4 (Ph), 128.6 (Ph), 128.5 (Ph), 126.0 (Ph), 128.0-102.0 (m, 2×CF₂,CF₃), 38.0 (CH₂), 35.8 (CH₂), 31.2 (CH₂), 28.4 (CH₂), 22.4 (CH₂); ¹⁹F NMR (CDCl₃): δ −9.3 (CF₃), −49.9 (CF₂), 55.4 (CF₂).

### Example 4

### Synthesis and Characterization of 1,1,1,2,2-pentafluoro-7-(4-hexyloxy-phenyl)-3-heptanone

### (2E,4E)-5-(4-Hexyloxy-phenyl)-penta-2,4-dienoic acid ethyl ester

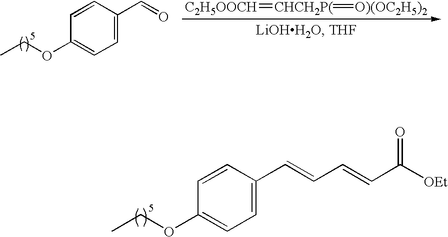

A solution of 4-hexyloxy-benzaldehyde (0.2 g, 1 mmol), triethyl 4-phosphonocrotonate (0.37 g, 1.5 mmol), lithium hydroxide (0.036 g, 1.5 mmol) and molecular sieves (1.5 g/mmol 4-hexyloxy-benzaldehyde) in dry tetrahydrofuran (10 mL) was refluxed under argon for 24 h. The reaction mixture was then cooled to room temperature, filtered through a thin pad of celite and the solvent evaporated under reduced pressure. The residual oil was purified by chromatography on silica gel eluting with ether/petroleum ether (1/9) to give 0.215 g (71%) of (2E,4E)-5-(4-hexyloxy-phenyl)-penta-2,4-dienoic acid ethyl ester as a white solid.

¹H NMR (CDCl₃): δ 7.48 (m, 1H, CH), 7.40 (d, J=8.8 Hz, 2H, Ph), 6.87 (d, J=8.8 Hz, 2H, Ph), 6.81 (m, 1H, CH), 6.71 (d, J=15.4 Hz, 1H, CH), 5.94 (d, J=15.4 Hz, 1H, CHCOO), 4.23 (q, J=7.4 Hz, 2H, OCH₂CH₃), 3.97 (t, J=6.2 Hz, 2H, CH₂O), 1.77 (m, 2H, CH₂CH₂O), 1.34 (m, 9H, 3×CH₂, CH₃), 0.92 (t, J=6.8 Hz, 3H, CH₃); ¹³C NMR (CDCl₃): δ 167.2 (COO), 160.0 (Ph), 145.0 (CH), 140.2 (CH), 131.9 (Ph), 128.6 (Ph), 123.9 (CH), 119.9 (CH), 114.7 (Ph), 68.0 (CH₂O), 60.2 (OCH₂CH₃), 31.6 (CH₂), 29.1 (CH₂), 25.7 (CH₂), 22.6 (CH₂), 14.3 (CH₃), 14.0 (CH₃).

### 5-(4-Hexyloxy-phenyl)-pentanoic acid ethyl ester

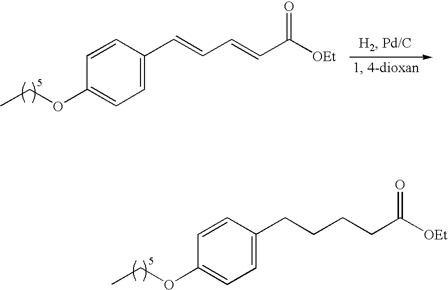

A solution of (2E,4E)-5-(4-hexyloxy-phenyl)-penta-2,4-dienoic acid ethyl ester (0.210 g, 0.7 mmol) in dry 1,4-dioxane (7 mL) containing 10% Pd/C (0.07 g) was stirred under H₂ for 12 h at room temperature. After filtration through a pad of celite, the solvent was removed in vacuo to give the title compound (0.212 g, 0.7 mmol) as a colorless oil.

¹H NMR (CDCl₃): δ 7.07 (d, J=8.8 Hz, 2H, Ph), 6.81 (d, J=8.8 Hz, 2H, Ph), 4.12 (q, J=7 Hz, 2H, OCH₂CH₃), 3.92 (t, J=6.6 Hz, 2H, CH₂O), 2.57 (t, J=7 Hz, 2H, CH₂), 2.31 (t, J=6.8 Hz, 2H, CH₂COO), 1.58 (m, 6H, 3×CH₂), 1.36 (m, 9H, 3×CH₂, CH₃), 0.92 (t, J=6.8 Hz, 3H, CH₃); ¹³C NMR (CDCl₃): δ 173.3 (CO), 157.1 (Ph), 133.7 (Ph), 128.9 (Ph), 114.1 (Ph), 67.7 (CH₂O), 59.9 (OCH₂CH₃), 34.4 (CH₂), 33.9 (CH₂), 31.4 (CH₂), 30.9 (CH₂), 29.1 (CH₂), 25.6 (CH₂), 24.3 (CH₂), 22.4 (CH₂), 14.0 (CH₃), 13.8 (CH₃).

### 5-(4-Hexyloxy-phenyl)-pentanoic acid

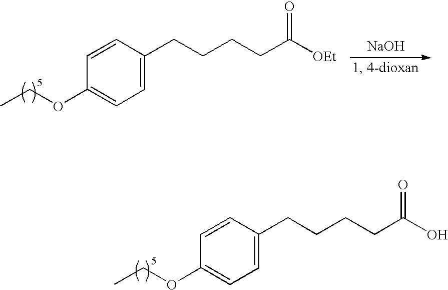

A solution of 5-(4-hexyloxy-phenyl)-pentanoic acid ethyl ester (0.212 g, 0.7 mmol) in methanol (1.4 mL) was treated with sodium hydroxide 1N (1 mmol). The mixture was stirred for 12 h at room temperature, acidified with hydrochloric acid 1N and extracted with ethyl acetate (3×10 mL). The solvent was removed in vacuo to afford 0.187 g (96%) of 5-(4-hexyloxy-phenyl)-pentanoic acid as a white solid.

¹H NMR (CDCl₃): δ 7.03 (d, J=8.4 Hz, 2H, Ph), 6.77 (d, J=8.4 Hz, 2H, Ph), 3.88 (t, J=6.2 Hz, 2H, CH₂O), 2.52 (t, J=6.8 Hz, 2H, CH₂), 2.32 (t, J=6.7 Hz, 2H, CH₂COO), 1.72 (m, 2H, CH₂), 1.61 (m, 4H, 2×CH₂), 1.31 (m, 6H, 3×CH₂), 0.89 (t, J=6.7 Hz, 3H, CH₃); ¹³C NMR (CDCl₃): δ 180.1 (COOH), 157.3 (Ph), 133.8 (Ph), 129.2 (Ph), 114.4 (Ph), 68.0 (CH₂O), 34.6 (CH₂), 33.9 (CH₂), 31.6 (CH₂), 31.0 (CH₂), 29.3 (CH₂), 25.7 (CH₂), 24.2 (CH₂), 22.6 (CH₂), 14.0 (CH₃).

1,1,1,2,2-pentafluoro-7-(4-hexyloxy-phenyl)-3-heptanone (FKGK15) was prepared according to the general method described in Example 2.

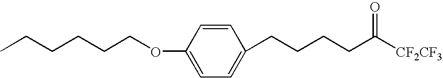

Yellow oil, yield 61%; ¹H NMR (CDCl₃): δ 7.06 (d, J=8 Hz, 2H, Ph), 6.81 (d, J=8 Hz, 2H, Ph), 3.93 (t, J=6.6 Hz, 2H, CH₂), 2.75 (t, J=6.6 Hz, 2H, CH₂), 2.57 (t, J=6.2 Hz, 2H, CH₂), 1.70 (m, 6H, 3×CH₂), 1.40 (m, 6H, 3×CH₂), 0.90 (t, J=6.8 Hz, 3H, CH₃); ¹³C NMR (CDCl₃): δ 194.2 (t, J_{CCF2}=26 Hz, CO), 157.4 (Ph), 133.4 (Ph), 129.1 (Ph), 117.8 (qt, J_{CF3}=287 Hz, J_{CCF2}=34 Hz, CF₃), 114.4 (Ph), 106.8 (tq, J_{CF2}=267 Hz, J_{CCF3}=38 Hz, CF₂), 68.0 (CH₂O), 37.2 (CH₂), 34.6 (CH₂), 31.6 (CH₂), 30.6 (CH₂), 29.3 (CH₂), 25.7 (CH₂), 22.6 (CH₂), 21.9 (CH₂), 14.0 (CH₃); ¹⁹F NMR (CDCl₃, TFA): δ −4.1 (CF₃), −45.6 (CF₂); MS (ESI) m/z (%): 379 (M⁻,100).

1,1,1-Trifluoro-6-(4hexyloxy-phenyl)-2-hexanone (FKGK2) was prepared according to the general method described in Example 2.

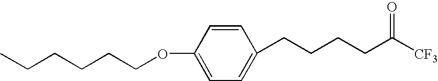

1,1,1-Trifluoro-6-(4-decyloxy-phenyl)-2-hexanone (FKGK4) was prepared following similar procedures.

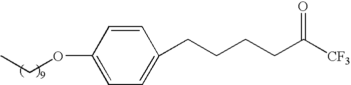

Yellowish oil, yield 46%. ¹H NMR (CDCl₃): δ 7.08 (d, J=8.6 Hz, 2H, Ph), 6.84 (d, J=8.6 Hz, 2H, Ph), 3.94 (t, J=6.6 Hz, 2H, CH₂O), 2.73 (t, J=6.6 Hz, 2H, CH₂), 2.59 (t, J=7 Hz, 2H, CH₂), 1.73 (m, 6H, 3×CH₂), 1.34 (m, 14H, 7×CH₂), 0.90 (t, J=6.8 Hz, 3H, CH₃). ¹³C NMR (CDCl₃): δ 191.6 (q, J_{CCF}=35 Hz, COCF₃), 157.7 (Ph), 133.7 (Ph), 129.4 (Ph), 114.6 (Ph), 115.8 (q, J_{CF}=292 Hz, CF₃), 68.2 (CH₂O), 36.4 (CH₂), 34.8 (CH₂), 32.1 (CH₂), 30.9 (CH₂), 29.8 (CH₂), 29.8 (CH₂), 29.6 (CH₂), 29.5 (CH₂), 26.6 (CH₂), 26.3 (CH₂), 22.9 (CH₂), 22.1 (CH₂), 14.32 (CH₃). ¹⁹F NMR (CDCl₃, TFA): δ −1.6. MS (FAB) m/z (%): 386 (M⁻,100), 418 (22).

### Example 5

### Synthesis and Characterization of 1,1,1,2,2-Pentafluoro-6-(4-octyl-phenoxy)-hexan-3-one

### 4-(4-Octyl-phenoxy)-butyric acid ethyl ester

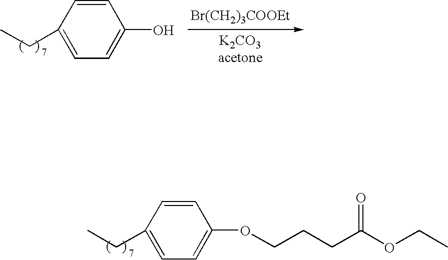

A solution of 4-octyl-phenol (1.0 g, 4.8 mmol), K₂CO₃ (1.99 g, 14.4 mmol) and ethyl 4-bromobutyrate (1.12 gr, 5.76 mmol) in acetone (38.4 mL) was refluxed for 24 h. The reaction mixture was then cooled to room temperature and the solvent was removed under reduced pressure. The residual oil was purified by column chromatography on silica gel eluting with ethyl acetate/petroleum ether (1/9) to give 1.09 g (71%) of 4-(4-octyl-phenoxy)-butyric acid ethyl ester as a colorless oil.

¹H NMR (CDCl₃): δ 7.07 (d, J=8.8 Hz, 2H, Ph), 6.81 (d, J=8.8 Hz, 2H, Ph), 4.17 (q, J=7 Hz, 2H, OCH₂CH₃), 3.92 (t, J=6.6 Hz, 2H, CH₂O), 2.52 (m, 4H, 2×CH₂), 2.10 (m, 2H, CH₂CH₂COO), 1.58 (m, 2H, CH₂), 1.29 (br, 13H, 5×CH₂, CH₃), 0.90 (t, J=6.8 Hz, 3H, CH₃); ¹³C NMR (CDCl₃): δ 173.5 (CO), 157.1 (Ph), 135.3 (Ph), 129.5 (Ph), 114.5 (Ph), 66.9 (CH₂O), 60.5 (OCH₂CH₃), 35.3 (CH₂), 32.1 (CH₂), 32.0 (CH₂), 31.1 (CH₂), 29.8 (CH₂), 29.5 (CH₂), 25.0 (CH₂), 22.9 (CH₂), 14.5 (CH₃), 14.3 (CH₃).

### 4-(4-Octyl-phenoxy)-butyric acid

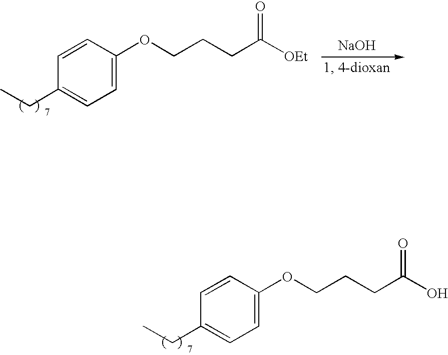

A solution of 4-(4-octyl-phenoxy)-butyric acid ethyl ester (1.09 g, 3.42 mmol) in 1,4-dioxane (4 mL) was treated with sodium hydroxide 1N (6 mmol). The mixture was stirred for 12 h at room temperature, acidified with hydrochloric acid 1N and extracted with ethyl acetate (3×15 mL). The solvent was removed in vacuo to afford 0.80 g (80%) of 4-(4-octyl-phenoxy)-butyric acid as a white solid (melting point (mp)=42-43° C.).

¹H NMR (CDCl₃): δ 7.09 (d, J=8.4 Hz, 2H, Ph), 6.82 (d, J=8.4 Hz, 2H, Ph), 4.00 (t, J=6.2 Hz, 2H, CH₂O), 2.58 (m, 4H, 2×CH₂), 2.10 (m, 2H, CH₂), 1.57 (m, 2H, CH₂), 1.28 (br, 10H, 5×CH₂), 0.89 (t, J=6.7 Hz, 3H, CH₃); ¹³C NMR (CDCl₃): δ 179.5 (COOH), 156.7 (Ph), 135.2 (Ph), 129.2 (Ph), 114.2 (Ph), 66.5 (CH₂O), 35.0 (CH₂), 31.9 (CH₂), 30.6 (CH₂), 30.5 (CH₂), 29.5 (CH₂), 29.4 (CH₂), 24.4 (CH₂), 22.6 (CH₂), 14.1 (CH₃).

1,1,1,2,2-Pentafluoro-6-(4-octyl-phenoxy)-hexan-3-one (FKGK16) was prepared according to the general method described in Example 2.

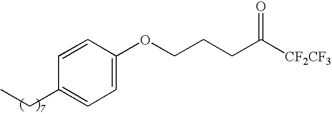

Yellowish oil, yield 70%; ¹H NMR (CDCl₃): δ 7.10 (d, J=8 Hz, 2H, Ph), 6.81 (d, J=8 Hz, 2H, Ph), 3.99 (t, J=6.6 Hz, 2H, CH₂), 3.00 (t, J=6.6 Hz, 2H, CH₂), 2.57 (t, J=6.2 Hz, 2H, CH₂), 2.17 (m, 2H, CH₂), 1.60 (m, 2H, CH₂), 1.30 (br, s, 10H, 5×CH₂), 0.91 (t, J=6.8 Hz, 3H, CH₃); ¹³C NMR (CDCl₃): δ 193.9 (t, J_{CCF2}=26 Hz, CO), 156.6 (Ph), 135.5 (Ph), 129.1 (Ph), 117.8 (qt, J_{CF3}=287 Hz, J_{CCF2}=34 Hz, CF₃), 114.4 (Ph), 106.8 (tq, J_{CF2}=267 Hz, J_{CCF3}=38 Hz, CF₂), 65.8 (CH₂O), 35.4 (CH₂), 34.6 (CH₂), 31.6 (CH₂), 29.7 (CH₂), 29.5 (CH₂), 29.3 (CH₂), 25.3 (CH₂), 22.7 (CH₂), 22.4 (CH₂), 14.3 (CH₃); ¹⁹F NMR (CDCl₃, TFA): δ −4.2 (CF₃), −45.6 (CF₂); MS (ESI) m/z (%): 393 (M³¹,100).

Following a similar procedure the corresponding trifluoromethyl ketone was prepared.

### 1,1,1-Trifluoro-5-(4-octyl-phenoxy)-pentan-2-one (FKGK6)

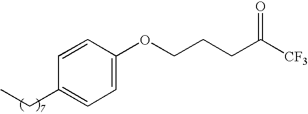

Yellowish oil, yield 32%; ¹H NMR (CDCl₃): δ 7.10 (d, J=8 Hz, 2H, Ph), 6.81 (d, J=8 Hz, 2H, Ph), 3.99 (t, J=6.6 Hz, 2H, CH₂), 2.95 (t, J=6.6 Hz, 2H, CH₂), 2.54 (t, J=6.2 Hz, 2H, CH₂), 2.16 (m, 2H, CH₂), 1.57 (m, 2H, CH₂), 1.26 (br, s, 10H, 5×CH₂), 0.88 (t, J=6.8 Hz, 3H, CH₃). ¹³C NMR (CDCl₃): δ 191.9 (t, J_{CCF2}=34 Hz, CO), 156.5 (Ph), 135.5 (Ph), 129.3 (Ph), 115.8 (q, J_{CF}=292 Hz, CF₃), 114.2 (Ph), 65.8 (CH₂O), 35.0 (CH₂), 33.1 (CH₂), 31.9 (CH₂), 31.7 (CH₂), 29.5 (CH₂), 29.3 (CH₂), 22.7 (CH₂), 22.4 (CH₂), 14.0 (CH₃); ¹⁹F NMR (CDCl₃, TFA): δ −1.5; MS (ESI) m/z (%): 343 (M⁻,100).

### Example 6

### Synthesis and Characterization of 1,1,1,2,2-Pentafluoro-5-(4-hexyloxy-phenyl)-3-pentanone

### (E)-3-(4hexyloxy-phenyl)-acrylic acid methyl ester

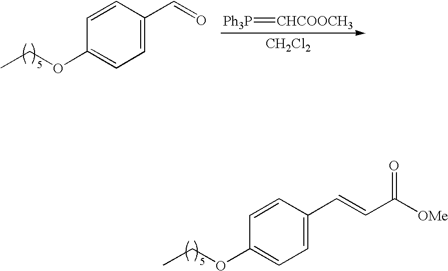

A solution of 4-hexyloxy-benzaldehyde (0.2 g, 1 mmol) and (triphenyl-phosphanylidene)-acetic acid methyl ester (0.334 g, 1 mmol) in dry dichloromethane (3 mL) was refluxed under argon for 24 h. The reaction mixture was then cooled to room temperature and the solvent evaporated under reduced pressure. The residual oil was purified by column chromatography on silica gel eluting with ethyl acetate/petroleum ether (1/9) to give 0.244 g (93%) of (E)-3-(4-hexyloxy-phenyl)-acrylic acid methyl ester as a white solid.

¹H NMR (CDCl₃): δ 7.63 (d, J=15.8 Hz, 1H, CH═CHCO), 7.43 (d, J=8.8 Hz, 2H, Ph), 6.87 (d, J=8.8 Hz, 2H, Ph), 6.28 (d, J=15.8 Hz, 1H, CHCOO), 3.95 (t, J=6.4 Hz, 2H, CH₂O), 3.77 (s, 3H, OCH₃), 1.76 (m, 2H, CH₂CH₂O), 1.34 (m, 6H, 3×CH₂), 0.89 (t, J=6.8 Hz, 3H, CH₃). ¹³C NMR (CDCl₃): δ 167.7 (COO), 161.0 (Ph), 144.6 (CH), 129.6 (Ph), 126.8 (Ph), 114.9 (CH), 114.7 (Ph), 68.1 (CH₂O), 51.5 (OCH₃), 31.5 (CH₂), 29.0 (CH₂), 25.6 (CH₂), 22.5 (CH₂), 13.9 (CH₃).

### 3-(4-Hexyloxy-phenyl)-propanoic acid

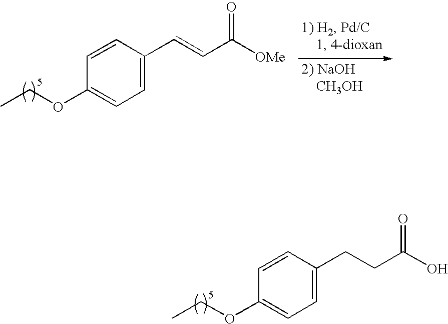

(E)-3-(4-Hexyloxy-phenyl)-acrylic acid methyl ester (0.244 g, 0.93 mmol) was reacted as described in Example 3 to give 0.21 g (90%) of the title material as a white solid (mp=70-72° C.).

¹H NMR (CDCl₃): δ 7.14 (d, J=8.2 Hz, 2H, Ph), 6.86 (d, J=8.2 Hz, 2H, Ph), 3.96 (t, J=6.6 Hz, 2H, CH₂O), 2.93 (t, J=7.8 Hz, 2H, CH₂), 2.67 (t, J=7.4 Hz, 2H, CH₂), 1.80 (t, J=6.6 Hz, 2H, CH₂), 1.39 (m, 6H, 3×CH₂), 0.92 (t, J=6.7 Hz, 3H, CH₃). ¹³C NMR (CDCl₃): δ 179.0 (COO), 157.6 (Ph), 132.0 (Ph), 129.1 (Ph), 114.5(Ph), 67.9 (CH₂O), 35.9 (CH₂), 31.5 (CH₂), 29.7 (CH₂), 29.2 (CH₂), 25.7 (CH₂), 22.5 (CH₂), 14.0 (CH₃).

1,1,1,2,2-Pentafluoro-5-(4-hexyloxy-phenyl)-3-pentanone (FKGK14) was prepared according to the general method described in Example 2.

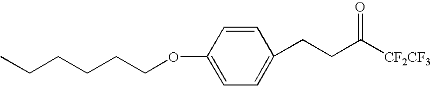

Yellow oil, yield 76%. ¹H NMR (CDCl₃): δ 7.10 (d, J=8.6 Hz, 2H, Ph), 6.85 (d, J=8.6 Hz, 2H, Ph), 3.94 (t, J=6.6 Hz, 2H, CH₂O), 3.02 (t, J=7 Hz, 2H, CH₂), 2.96 (t, J=6.4 Hz, 2H, CH₂), 1.80 (m, 2H, CH₂), 1.39 (m, 6H, 3×CH₂), 0.93 (t, J=6.4 Hz, 3H, CH₃). ¹³C NMR (CDCl₃): δ 193.5 (t, J_{CCF}=26 Hz, CO), 157.9 (Ph), 130.9 (Ph), 129.2 (Ph), 117.8 (qt, J_{CF3}=286 Hz, J_{CCF2}=34 Hz, CF₃), 114.7 (Ph), 106.8 (tq, J_{CF2}=265 Hz, J_{CCF3}=38 Hz, CF₂), 68.0 (CH₂O), 39.4 (CH₂), 31.6 (CH₂), 29.3 (CH₂), 27.5 (CH₂), 25.7 (CH₂), 22.6 (CH₂), 13.9 (CH₃). ¹⁹F NMR (CDCl₃): δ −4.2 (CF₃), −45.6 (CF₂). MS (ESI) m/z (%): 351 (M⁻,100), 350 (25).

1,1,1-Trifluoro-4-(4-hexyloxy-phenyl)-2-butanone (FKGK1) was prepared according to similar procedures.

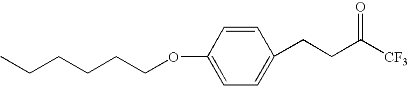

Yellowish oil, yield 53%. ¹H NMR (CDCl₃): δ 7.10 (d, J=8.6 Hz, 2H, Ph), 6.84 (d, J=8.6 Hz, 2H, Ph), 3.93 (t, J=6.2 Hz, 2H, CH₂), 2.97 (m, 4H, 2×CH₂), 1.77 (m, 2H, CH₂), 1.37 (m, 6H, 3×CH₂), 0.91 (t, J=6.6 Hz, 3H, CH₃). ¹³C NMR (CDCl₃): δ 190.7 (q, J_{CCF}=35 Hz, COCF₃), 157.9 (Ph), 131.0 (Ph), 129.2 (Ph), 114.6 (Ph), 115.5 (q, J_{CF}=292 Hz, CF₃), 67.9 (OCH₂), 38.3 (CH₂), 31.5 (CH₂), 29.2 (CH₂), 27.4 (CH₂), 25.6 (CH₂), 22.5 (CH₂), 13.9 (CH₃). ¹⁹F NMR (CDCl₃, TFA): δ −1.5. MS (ESI) m/z (%): 301 (M⁻,100).

1,1,1-Trifluoro-4-(4-decyloxy-phenyl)-2-butanone (FKGK3) was prepared according to similar procedures.

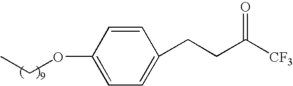

Yellowish oil, yield 46%. ¹H NMR (CDCl₃): δ 7.12 (d, J=8.6 Hz, 2H, Ph), 6.85 (d, J=8.6 Hz, 2H, Ph), 3.96 (t, J=6.6 Hz, 2H, CH₂O), 2.98 (m, 4H, 2×CH₂), 1.79 (m, 2H, CH₂), 1.31 (m, 14H, 7×CH₂), 0.92 (t, J=6.8 Hz, 3H, CH₃). ¹³C NMR (CDCl₃): δ 190.5 (q, J_{CCF}=35 Hz, COCF₃), 157.8 (Ph), 131.0 (Ph), 129.1 (Ph), 114.6 (Ph), 115.5 (q, J_{CF}=292 Hz, CF₃), 68.0 (CH₂O), 38.3 (CH₂), 31.8 (CH₂), 29.6 (CH₂), 29.3 (CH₂), 29.2 (CH₂), 27.4 (CH₂), 26.0 (CH₂), 22.6 (CH₂), 14.0 (CH₃). ¹⁹F NMR (CDCl₃, TFA): δ −1.5. MS (FAB) m/z (%): 358 (M⁺,80), 376 (8).

1,1,1-Trifluoro-4-(4-benzyloxy-phenyl)-2-butanone (FKGK5) was prepared according to similar procedures.

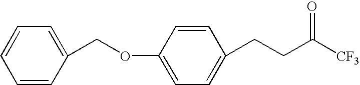

Yellowish solid, yield 43%, mp 71-72° C. ¹H NMR (CDCl₃): δ 8.42 (m, 5H, Ph), 7.15 (d, J=8.4 Hz, 2H, Ph), 6.95 (d, J=8.4 Hz, 2H, Ph), 5.07 (s, PhCH₂), 3.00 (m, 4H, 2×CH₂). ¹³C NMR (CDCl₃): δ 190.7 (q, J_{CCF}=35 Hz, COCF₃), 157.4 (Ph), 136.9 (Ph), 131.4 (Ph), 128.5 (Ph), 127.9 (Ph), 127.4 (Ph), 115.4 (q, J_{CF}=290 Hz, CF₃), 114.9 (Ph), 69.9 (CH₂O), 38.2 (CH₂), 27.4 (CH₂). ¹⁹F NMR (CDCl₃, TFA): δ −1.4. MS (ESI) m/z (%): 307 (M⁻,100), 243 (31).

### Example 7

### Synthesis and Characterization of 1,1,1,3-Tetrafluoro-6-phenyl-2-hexanone

### 2-Fluoro-5-phenyl-pentanoic acid methyl ester

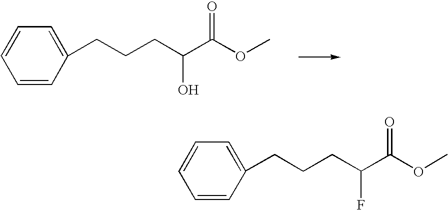

To a solution of bis(2-methoxyethyl)amino-sulfur-trifluoride, Deoxofluor (0.2 mL, 1 mmol) in dry dichloromethane (0.2 mL) at −78° C. was added 2-hydroxy-5-phenyl-pentanoic acid methyl ester (0.208 g, 1 mmol). After stirring for 2 h at −78° C. and another 3 h at room temperature, the reaction mixture quenched with saturated aqueous NaHCO₃ (2.5 mL). The organic phase was then washed with brine and dried (Na₂SO₄). The solvent was evaporated under reduced pressure and the residual oil was purified by column chromatography on silica gel eluting with ethyl acetate/petroleum ether (3/7) to give 0.127 g (60%) of 2-fluoro-5-phenyl-pentanoic acid methyl ester as a yellowish oil.

¹H NMR (CDCl₃): δ 7.22 (m, 5H, Ph), 4.91 (dt, J_{HF}=55.1, Hz, J_{HH}=5.9 Hz, 1H, CHFCO), 3.78 (s, 3H, CH₃O), 2.67 (t, J=6.6 Hz, 2H, PhCH₂), 2.05-1.69 (m, 4H, 2×CH₂); ¹³C NMR (CDCl₃): δ 170.2 (d, J_{CCF}=23 Hz, CO), 141.3 (Ph), 128.3 (Ph), 28.3 (Ph), 125.9 (Ph), 88.79 (d, J_{CF}=183 Hz, CHF), 52.1 (CH₃), 35.1 (CH₂), 31.8 (d, J_{CCF}=21 Hz, CH₂CHF), 25.9 (CH₂); ¹⁹F NMR (CDCl₃): δ −114.1 (m, CF). MS (ESI) m/z (%): 212 (M⁺,100).

### 1,1,1,3-tetrafluoro-6-phenyl-2-hexanone (FKGK9)

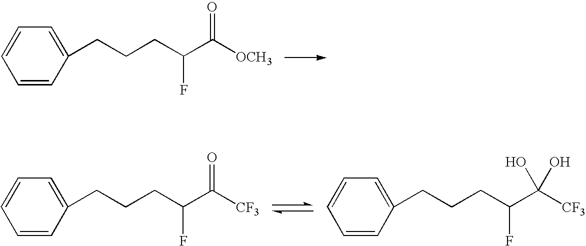

A solution of methyl 2-fluoro-5-phenyl-pentanoate (0.125 g, 0.6 mmol) and trifluoromethyltrimethylsilane (170 μL, 1.15 mmol) in ethylene glycol dimethyl ether (0.55 mL) at 0° C. was treated with cesium fluoride (3 mg). After stirring for 30 min at 0° C. and another 18 h at 25° C. the reaction mixture was treated with concentrated HCl (1 mL). After stirring for another 18 h at 25° C., the reaction mixture was diluted with ethyl acetate (10 mL). The organic phase was then washed with brine and dried (Na₂SO₄). The solvent was evaporated under reduced pressure and the residual oil was purified by column chromatography on silica gel eluting with ethyl acetate/petroleum ether (3/7) to give 0.068 g (47%) of 1,1,1,3-tetrafluoro-6-phenyl-2-hexanone as an yellowish oil.

¹H-NMR (CDCl₃): δ 7.25 (m, 5H, Ph), 5.20 (dt, J_{HF}=48.2 Hz, J_{HH}=6.2 Hz, 1/3H, CH), 4.65 (dt, J_{HF}=48.2 Hz, J_{HH}=6.2 Hz, 2/3H, CH), 3.85 (b, 2/3H, OH), 3.62 (br, 2/3H, OH), 2.68 (t, J=6.2 Hz, 2H, CH₂), 2.05-1.67 (m, 4H, 2×CH₂). ¹³C NMR (CDCl₃): 141.5 (Ph), 128.4 (Ph), 126.1 (Ph), 125.9 (Ph), 122.6 (q, J_{CF3}=286 Hz, CF₃), 92.4 (d, J_{CF}=175 Hz, CF), 35.3 (CH₂), δ 31.8 (d, J_{CF}=20 Hz, CH₂), 27.6 (d, J_{CF}=20 Hz, CH₂). ¹⁹F NMR (CDCl₃, TFA): δ 1.6 (CF), −5.3 (CF₃).

1,1,1,3-Tetrafluoro-2-heptadecanone (FKGK8) was prepared by similar reactions.

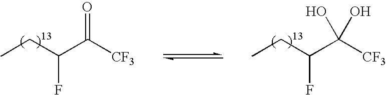

White solid, yield 58%. ¹H-NMR (CDCl₃): δ 5.23 (dt, J_{HF}=48.2 Hz, J_{HH}=6.2 Hz, 1/3H, CH), 4.65 (dt, J_{HF}=49.4 Hz, J_{HH}=6.6 Hz, 2/3H, CH), 3.74 (s, 2/3H, OH), 3.49 (s, 2/3H, OH), 2.08-1.27 (m, 26H, 13×CH₂), 0.89 (t, J=7 Hz, 3H, CH₃). ¹³C NMR (CDCl₃): δ 122.6 (q, J_{CF3}=286 Hz, CF₃), 92.9 [C(OH)₂], 92.4 (d, J_{CF}=185 Hz, CF), 32.1 (CH₂), 31.6 (d, J_{CF}=20 Hz, CH₂), 29.9 (CH₂), 29.7 (CH₂), 29.6 (CH₂), 29.5 (CH₂), 29.4 (CH₂), 29.1 (CH₂), 28.5 (CH₂), 28.1 (CH₂), 22.7 (CH₂), 22.3 (CH₂), 14.3 (CH₃). ¹⁹F NMR (CDCl₃, TFA): δ 1.6 (CF), −5.3 (CF₃). MS (ESI) m/z (%): 343 (M⁻,100).

### 1,1,1,3-Tetrafluoro-7-phenyl-2-heptanone (FKGK10)

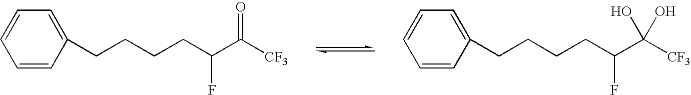

A solution of methyl 2-fluoro-6-phenyl-hexanoate (0.054 g, 0.24 mmol) and trifluoromethyltrimethylsilane (245 μL, 1.66 mmol) in toluene (2.2 mL) at −78° C. was treated with tetrabutylammonium fluoride (1.0 M in THF, 11 μL, 0.011 mmol). The cooling bath was removed, and the reaction mixture was stirred at room temperature for 2 h and concentrated in vacuo. The reaction mixture was diluted in THF (1.9 mL) and then treated with a mixture of tetrabutylammonium fluoride and glacial acetic acid (0.24 mmol). After stirring for 1 h at room temperature, ethyl acetate was added and the mixture was washed with saturated.aqueous Na₂CO₃, brine, dried (Na₂SO₄) and concentrated in vacuo. The residue was purified by column chromatography on silica gel eluting with petroleum ether/ethyl acetate (7:3) to give the title compound (59 mg, 94%) as pale yellow oil.

¹H-NMR (CDCl₃): δ 7.25 (m, 5H, Ph), 5.22 (dt, J_{HF}=43.8 Hz, J_{HH}=6.3 Hz, 1/3H, CH), 4.65 (dt, J_{HF}=48.2 Hz, J_{HH}=6.2 Hz, 2/3H, CH), 2.64 (t, J=6.2 Hz, 2H, CH₂), 2.05-1.40 (m, 6H, 3×CH₂). ¹³C NMR (CDCl₃): 141.1 (Ph), 128.3 (Ph), 125.8 (Ph), 125.7 (Ph), 121.8 (q, J_{CF}=286 Hz, CF₃), 92.4 (d, J_{CF}=175 Hz, CF), 35.6 (CH₂), δ 31.8 (d, J_{CF}=20 Hz, CH₂), 27.6 (d, J_{CF}=20 Hz, CH₂), 20.9 (CH₂). ¹⁹F NMR (CDCl₃, TFA): δ −4.7 (CF), −11.6 (CF₃).

### Example 8

### Synthesis and Characterization of 1,1,1,3,3-Pentafluoro-6-phenyl-hexan-2-one (FKGK7)

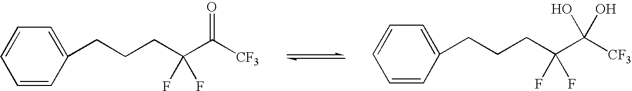

A solution of ethyl-2,2-difluoro-5-phenyl-pentanoate (0.100 g, 0.42 mmol) and trifluoromethyltrimethylsilane (80 μL, 0.55 mmol) in ethylene glycol dimethyl ether (0.55 mL) at 0° C. was treated with cesium fluoride (2 mg). After stirring for 30 min at 0° C. and another 18 h at room temperature, the reaction mixture was treated with concentrated HCl (1 mL). The reaction mixture was stirred for another 18 h at room temperature and then was diluted with ethyl acetate (10 mL). The organic phase was washed with brine and dried (Na₂SO₄). The solvent was evaporated under reduced pressure and the residual oil was purified by column chromatography on silica gel eluting with ethyl acetate/petroleum ether (3/7) to give 0.032 g (35%) of 1,1,1,3,3-pentafluoro-6-phenyl-hexan-2-one as an yellowish oil.

¹H NMR (CDCl₃): δ 7.20 (m, 5H, Ph), 3.93 (br, 2H, 2×OH), 2.69 (t, J=7.6 Hz, 2H, PhCH₂), 2.22-1.88 (m, 4H, 2×CH₂). ¹³C NMR (CDCl₃): δ 141.2 (Ph), 128.4 (Ph), 126.3 (Ph), 126.0 (Ph), 121.5 (q, J=286 Hz, CF₃), 120.7 (t, J=249 Hz, CF₂), 92.3 [C(OH)₂], 35.2 (CH₂), 30.8 (t, J_{CCF2}=23 Hz, CH₂CF₂), 22.5 (t, J_{CCCF2}=4 Hz, CH₂CH₂CF₂). ¹⁹F NMR (CDCl₃, TFA): δ −3.2 (CF₃), −36.4 (CF₂). Ms (ESI) m/z (%): 283 (M⁻,65), 213 (100).

### Example 9

### Synthesis of FKGK11 using the N-methoxy-N-methyl amide

### N-Methoxy-N-methyl-5-phenylpentanamide

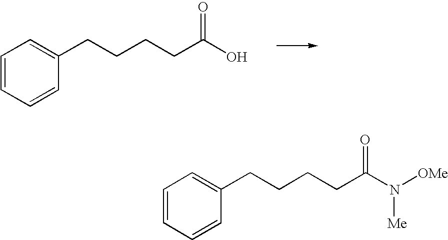

To a stirring solution of 5-phenylpentanoic acid (1.68 mmol) in CH₂Cl₂ (9 mL) at room temperature, DMAP (1.68 mmol) was added followed by MeONHMe.HCl (1.68 mmol), N-methyl morpholine (1.68 mmol) and WSCl.HCl (1.68 mmol). The reaction mixture was stirred for 16 h. It was then washed with 1N HCl (3×10 mL), 5% aq. solution of NaHCO₃ (3×10 mL), brine (10 mL) and dried over MgSO₄. The organic solvent was evaporated in vacuo to receive the desired product as colourless oil. Yield: 82%.

¹H NMR (CDCl₃): 7.37-7.03 (m, 5H, Ph), 3.63 (s, 3H, OCH₃), 3.16 (s, 3H, NCH₃), 2.61 (t, J=6.2 Hz, 2H, CH₂), 2.39 (t, J=6.6 Hz, 2H, CH₂), 1.74-1.57 (m, 4H, 2×CH₂). ¹³C NMR (CDCl₃): 174.6 (CO), 142.6 (Ph), 128.6 (Ph), 128.5 (Ph), 125.9 (Ph), 61.4 (OCH₃), 35.9 (NCH₃), 32.4 (CH₂), 31.9 (CH₂), 31.4 (CH₂), 24.5 (CH₂).

### 1,1,1,2,2-Pentafluoro-7-phenyl-3-heptanone (FKGK11)

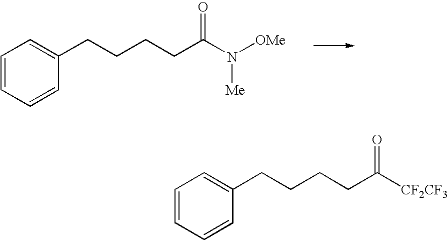

To a stirring solution of N-methoxy-N-methyl amide (0.36 mmol) in Et₂O (5 mL) at −78° C., pentafluoroiodoethane (1.80 mmol) was added followed by drop-wise addition of MeLi.LiBr solution 1M in ether (1.80 mmol). The reaction mixture was stirred at −78° C. and monitored by TLC. Once the reaction was finished (90 min), the reaction mixture was poured into H₂O and acidified with a 10% solution of KHSO₄ (pH 5). The layers were separated and the aqueous layer was extracted with Et₂O (3×15 mL). The combined organic layers were washed with a 5% aq. solution of NaHCO₃ (40 mL) and dried over MgSO₄. The organic solvent was evaporated in vacuo and the residue was purified by column chromatography eluting with the appropriate petroleum ether: EtOAc mixture. Yield 98%.

### Example 10

### Synthesis of FKGK11 using the Symmetric Anhydride

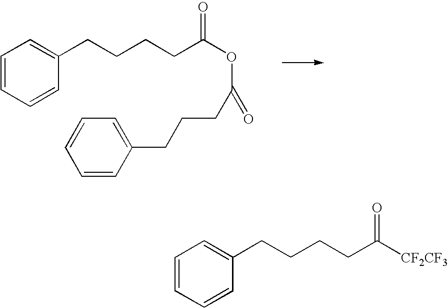

To a stirring solution of the 5-phenylpentanoic acid (1.68 mmol) in THF (2 mL) at 0° C., DCC (2.02 mmol) was added and left stirring for 1 h. The reaction mixture was filtered and the filtrate was concentrated. The residue was dissolved in Et₂O (10 mL) and cooled at −78° C. Pentafluoroiodoethane (3.78 mmol) was added followed by drop-wise addition of MeLi.LiBr solution 1M in ether (3.78 mmol). The reaction mixture was stirred at −78° C. and monitored by TLC. Once the reaction was finished (90 min), the reaction mixture was poured into H₂O and acidified with a 10% solution of KHSO₄ (pH 5). The layers were separated and the aqueous layer was extracted with Et₂O (3×15 mL). The combined organic layers were washed with a 5% aq. solution of NaHCO₃ (40 mL) and dried over MgSO₄. The organic solvent was evaporated in vacuo and the residue was purified by column chromatography eluting with the appropriate petroleum ether:EtOAc mixture. Yield 45%.

### Example 11

### Synthesis of FKGK11 using the Morpholino Amide

### 1-Morpholino-5-phenylpentan-1-one

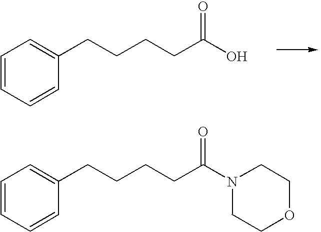

To a stirring solution of 5-phenylpentanoic acid (2.60 mmol) in CH₂Cl₂ (15 mL) at room temperature, DMF (0.1 mL) was added followed by oxalyl chloride (7.60 mmol). The reaction mixture was stirred for 3 h. The organic solvents were removed in vacuo and Et₂O (15 mL) was added at 0° C. Pyridine (15.6 mmol) was added drop-wise, followed by drop-wise addition of morpholine (15.5 mmol) and left stirring for 14 h. Then, water (10 mL) was added and stirred for 30 min at room temperature. The reaction mixture was washed with 1N HCl (3×10 mL), 5% aq. solution of NaHCO₃ (3×10 mL), brine (10 mL) and dried over MgSO₄. The organic solvent was evaporated in vacuo and the residue was purified by column chromatography eluting with a mixture of petroleum ether:AcOEt 7:3 to receive the desired product as yellowish oil. Yield: 91%

¹H NMR (CDCl₃): 7.36-7.04 (m, 5H, Ph), 3.67-3.49 (m, 6H, 3×CH₂), 3.41-3.24 (m, 2H, CH₂), 2.61 (t, J=6.3 Hz, 2H, CH₂), 2.26 (t, J=6.5 Hz, 2H, CH₂), 1.76-1.47 (m, 4H, 2×CH₂). ¹³C NMR (CDCl₃): 171.7 (CO), 142.4 (Ph), 128.6 (Ph), 128.5 (Ph), 125.9 (Ph), 67.1 (OCH₂), 66.8 (OCH₂), 46.2 (NCH₂) 42.0 (NCH₂), 35.9 (CH₂), 33.1 (CH₂), 31.3 (CH₂), 25.0 (CH₂).

### 1,1,1,2,2-Pentafluoro-7-phenyl-3-heptanone (FKGK11)

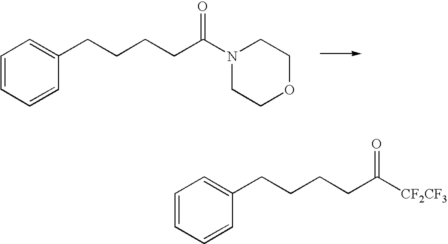

To a stirring solution of morpholino amide (0.36 mmol) in Et₂O (5 mL) at −78° C., pentafluoroiodoethane (1.80 mmol) was added followed by drop-wise addition of MeLi.LiBr solution 1M in ether (1.80 mmol). The reaction mixture was stirred at −78° C. and monitored by TLC. Once the reaction was finished (120 min), the reaction mixture was poured into H₂O and acidified with a 10% solution of KHSO₄ (pH 5). The layers were separated and the aqueous layer was extracted with Et₂O (3×15 mL). The combined organic layers were washed with a 5% aq. solution of NaHCO₃ (40 mL) and dried over MgSO₄. The organic solvent was evaporated in vacuo and the residue was purified by column chromatography eluting with the appropriate petroleum ether:EtOAc mixture. Yield 84%.

### Example 12

### Synthesis of FKGK11 Starting from the Aldehyde

### 1,1,1,2,2-Pentafluoro-7-phenylheptan-3-ol

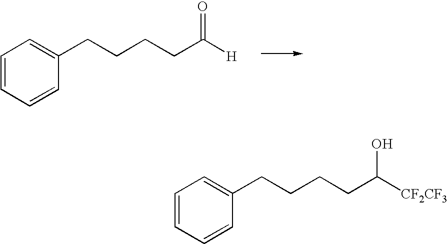

To a stirring solution of 5-phenylpantanal (0.54 mmol) in Et₂O (10 mL) at −78° C., pentafluoroiodoethane (2.43 mmol) was added followed by drop-wise addition of MeLi.LiBr solution 1M in ether (2.43 mmol). The reaction mixture was stirred at −78° C. and monitored by TLC. Once the reaction was finished, the reaction mixture was poured into H₂O and acidified with a 10% solution of KHSO₄ (pH 5). The layers were separated and the aqueous layer was extracted with Et₂O (3×15 mL). The combined organic layers were washed with a 5% solution of aq. NaHCO₃ (40 mL) and dried over MgSO₄. The organic solvent was evaporated in vacuo and the residue was purified by column chromatography eluting with the appropriate petroleum ether:EtOAc mixture. Yield 98%.

¹H NMR (CDCl₃): 7.26 (m, 5H, Ph), 4.01 (m, 1H, CH), 2.67 (t, J=7.0 Hz, 2H, PhCH₂), 1.82-1.18 (m, 6H, m, 3×CH₂). ¹⁹F NMR (CDCl₃): −10.2 (CF₃), −52.6(CFF), −59.3 (CFF).

### 1,1,1,2,2-Pentafluoro-7-phenyl-3-heptanone (FKGK11)

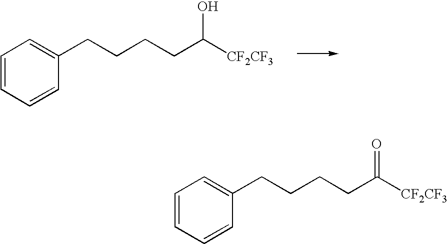

1,1,1,2,2-Pentafluoro-7-phenylheptan-3-ol (1 mmol) was dissolved in dichloromethane (15 mL) and treated with Dess-Martin periodinane (1.2 equiv) for 40 min. The organic phase was washed with brine and dried (Na₂SO₄). The solvent was evaporated under reduced pressure and the residual oil was purified by column chromatography on silica gel eluting with ethyl acetate/petroleum ether (5/95). Yield 84%.

### Example 13

### Synthesis of FKGK19 using N-methoxy-N-methyl amide

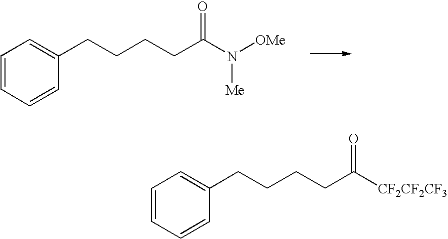

To a stirring solution of N-methoxy-N-methyl amide (0.36 mmol) in Et₂O (5 mL) at −78° C., heptafluoroiodoethane (1.80 mmol) was added followed by drop-wise addition of MeLi.LiBr solution 1M in ether (1.80 mmol). The reaction mixture was stirred at −78° C. and monitored by TLC. Once the reaction was finished (90 min), the reaction mixture was poured into H₂O and acidified with a 10% solution of KHSO₄ (pH 5). The layers were separated and the aqueous layer was extracted with Et₂O (3×15 mL). The combined organic layers were washed with a 5% aq. solution of NaHCO₃ (40 mL) and dried over MgSO₄. The organic solvent was evaporated in vacuo and the residue was purified by column chromatography eluting with the appropriate petroleum ether:EtOAc mixture. Yield 99%.

### Example 14

### Synthesis and Characterization of FKGK22

### 2-Fluoro-N-methoxy-N-methyl-5-phenylpentanamide

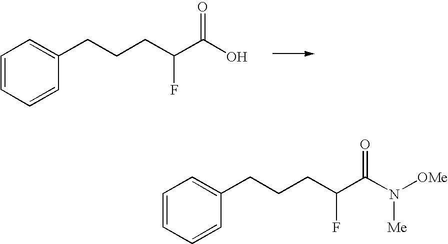

To a stirring solution of 2-fluoro-5-phenylpentanoic acid (0.38 mmol) in CH₂Cl₂ (4 mL) at room temperature, DMAP (0.38 mmol) was added followed by MeONHMe.HCl (0.38 mmol), N-methyl morpholine (0.38 mmol) and WSCl.HCl (0.38 mmol). The reaction mixture was stirred for 16 h. It was then washed with 1N HCl (3×5 mL), 5% aq. solution of NaHCO₃ (3×5 mL), brine (5 mL) and dried over MgSO₄. The organic solvent was evaporated in vacuo to receive the desired product as colourless oil. Yield: 77%.

¹H NMR (CDCl₃): 7.35-7.03 (m, 5H, Ph), 5.21 (m, 1H, CHF), 3.61 (s, 3H, OCH₃), 3.18 (s, 3H, NCH₃), 2.63 (t, J 6.7 Hz, 2H, CH₂), 2.01-1.62 (m, 4H, 2×CH₂). ¹³C NMR (CDCl₃): 171.8 (CO), 141.8 (Ph), 128.7 (Ph), 128.6 (Ph), 126.2 (Ph), 88.9 (CF), 61.7 (OCH₃), 35.4 (NCH₃), 32.3 (CH₂), 31.9 (CH₂), 31.4 (CH₂), 26.3 (CH₂). ¹⁹F NMR (CDCl₃): −126.9 (CF).

### 1,1,1,2,2,3,3,5-Octafluoro-8-phenyloctan-4-one

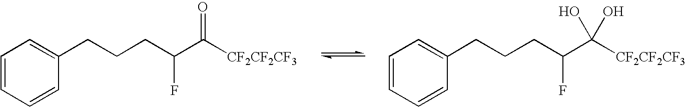

To a stirring solution of 2-fluoro-N-methoxy-N-methyl-5-phenylpentanamide (0.21 mmol) in Et₂O (4 mL) at −78° C., heptafluoroiodoethane (1.05 mmol) was added followed by drop-wise addition of MeLi.LiBr solution 1M in ether (1.05 mmol). The reaction mixture was stirred at −78° C. and monitored by TLC. Once the reaction was finished (90 min), the reaction mixture was poured into H₂O and acidified with a 10% solution of KHSO₄ (pH 5). The layers were separated and the aqueous layer was extracted with Et₂O (3×15 mL). The combined organic layers were washed with a 5% aq. solution of NaHCO₃ (40 mL) and dried over MgSO₄. The organic solvent was evaporated in vacuo and the residue was purified by column chromatography eluting with the appropriate petroleum ether:EtOAc mixture. Yield 81%.

¹H NMR (CDCl₃): 7.17 (m, 5H, Ph), 5.25 (1H, ddd, J=48.0, 8.2 and 3.9 Hz, 1H, CHF), 2.68 (m, 2H, CH₂), 2.01-1.75 (m, 4H, 2×CH₂). ¹³C NMR (CDCl₃): 190.5 (ddd, J=27.3, 25.6 and 22.3 Hz, CO), 141.0 (Ph), 128.7 (Ph), 128.6 (Ph), 126.4 (Ph), 119.5-105.6 (m, 2×CF₂, CF₃), 92.3 (d, J=187 Hz, CF), 35.2 (CH₂), 30.9 (d, J=21.2 Hz, CH₂), 26.2 (CH₂). ¹⁹F NMR (CDCl₃): −15.7 (CF₃), −54.7 (CF₂), −61.3 (CF₂), −131.5 (CF).

### Example 15

### mRNA Expression of PLA₂ Isoforms at Various Stages of EAE

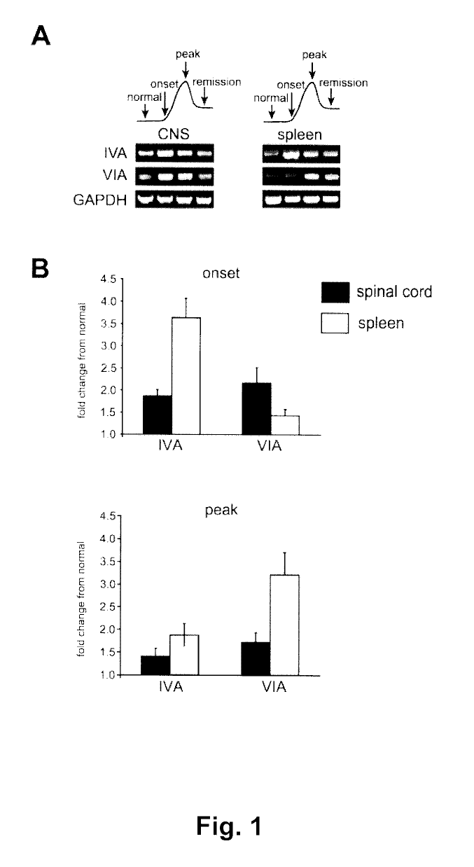

The mRNA expression of cPLA₂ (group IVA) and iPLA₂ (group VIA) in the spleen and spinal cord of SJL/J mice was assessed by RT-PCR at the onset, peak and remission stages of EAE. The mRNA expression of cPLA₂ type IVA is increased at the onset of EAE in both the spleen and spinal cord (FIG. 1), suggesting it is involved in initiation of the inflammatory changes in EAE. iPLA₂ (type VIA) mRNA levels increase at the clinical onset and the peak stages of EAE (FIG. 1) suggesting that it may be involved not only in the onset but also the progression of the disease.

### Example 16

### Protein Expression of PLA₂ Isoforms at Various Stages of EAE

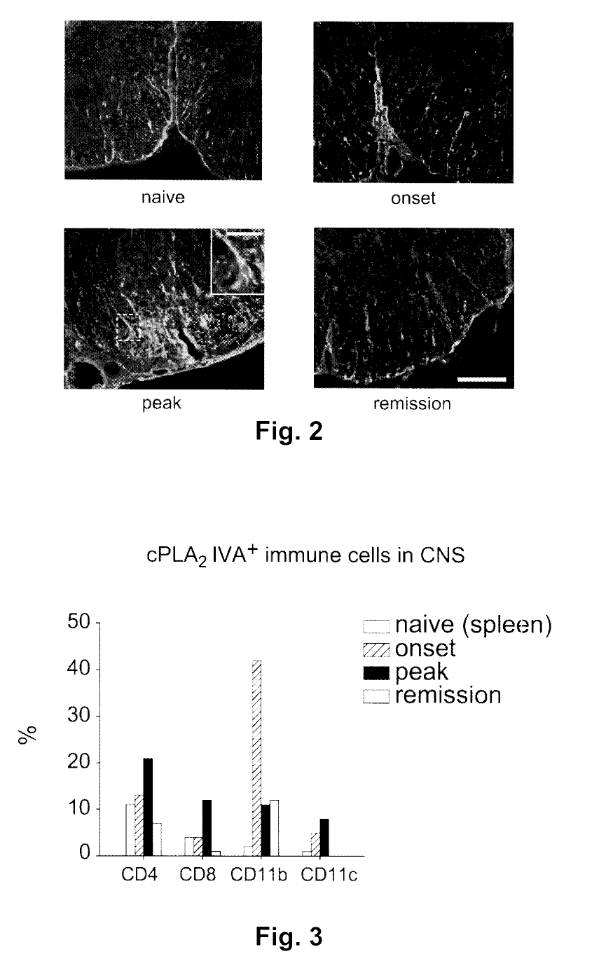

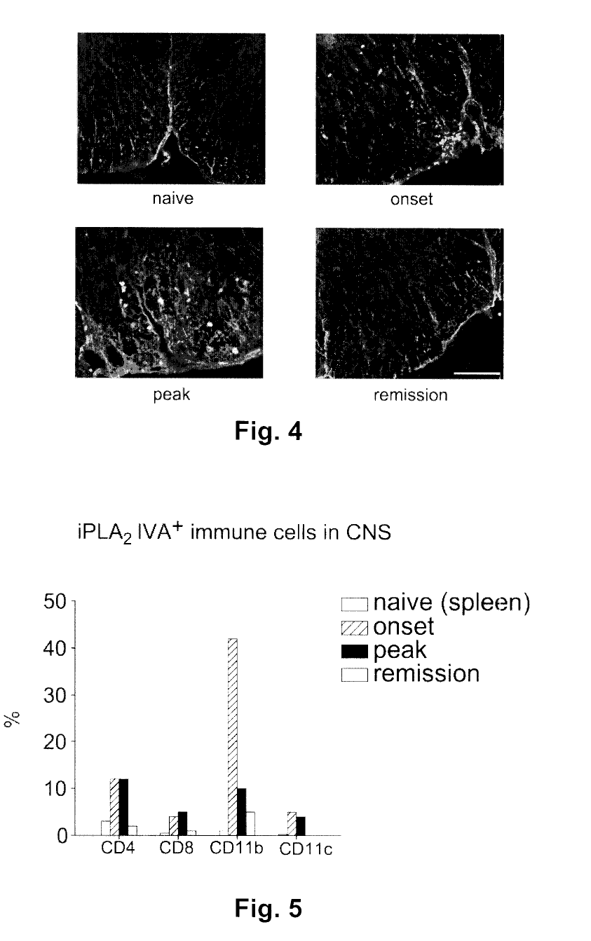

We next examined the protein expression of these PLA₂'s in the CNS by double immunofluorescence labelling in spinal cord tissue sections (FIGS. 2 and 4). The different types of immune cells that express these enzymes in the CNS were also analyzed through flow cytometry (FIGS. 3 and 5). cPLA₂ (group IVA) had low constitutive expression in oligodendrocytes, as well as some large motor neurons, as seen in the spinal cord of naïve animals. As immune cells entered the CNS in the onset of disease, about 64% of these cells were cPLA₂⁺ (FIG. 3). This expression was predominately by macrophages (CD11b⁺, 42%), followed by CD4⁺ T cells (14%), dendritic cells (CD11c⁺, 5%), and CD8⁺ T cells (4%). Of these cells, 40-50% of the CD4⁺ and CD8⁺ T cells were expressing cPLA₂, while ˜90% of the macrophages and dendritic cells were cPLA₂⁺. As the disease progressed, more cells began to enter the CNS, and those cells positive for cPLA₂ increased to 73%. Reactive astrocytes in and surrounding the EAE lesions within the CNS also began to express cPLA₂ at this peak stage (FIG. 2). As animals began to recover in the remission period, immune cells decreased their expression of cPLA₂ to 26%, and CNS cells returned to basal levels (FIG. 2).

On the other hand, iPLA₂ (group VIA) had only low constitutive expression in oligodendrocytes. As immune cells began to infiltrate the spinal cord at the onset of disease, the expression of iPLA₂ increased. This expression however was isolated to the infiltrating immune cells in EAE lesions, and not in CNS cells (FIG. 5). 66% of the immune cells were expressing iPLA₂ at early stages, 12% of which were CD4⁺ T cells, 4% were CD8⁺ T cells, 42% were CD11b⁺ macrophages, and 4% were CD11c⁺ dendritic cells (FIG. 5). Of these cells, 41% of CD4⁺ T cells, 44% of CD8⁺ T cells, 95% of macrophages, and 78% of dendritic cells were expressing iPLA₂. As a greater number of immune cells continued to enter the CNS at peak stage, iPLA₂ expression still remained high in these cells. The expression in the T cells and dendritic cells remained the same, while the proportion of macrophages decreased to 69%. The expression of iPLA₂ diminished back to very low levels at remission stage, where only 11% of immune cells were expressing iPLA₂ (2% of which were CD4⁺ T cells, 1% CD8⁺ T cells and 3% macrophages) (FIG. 5).

### Example 17

### Effect of Treatment with Specific PLA₂ Inhibitors on the Onset and Progression of EAE

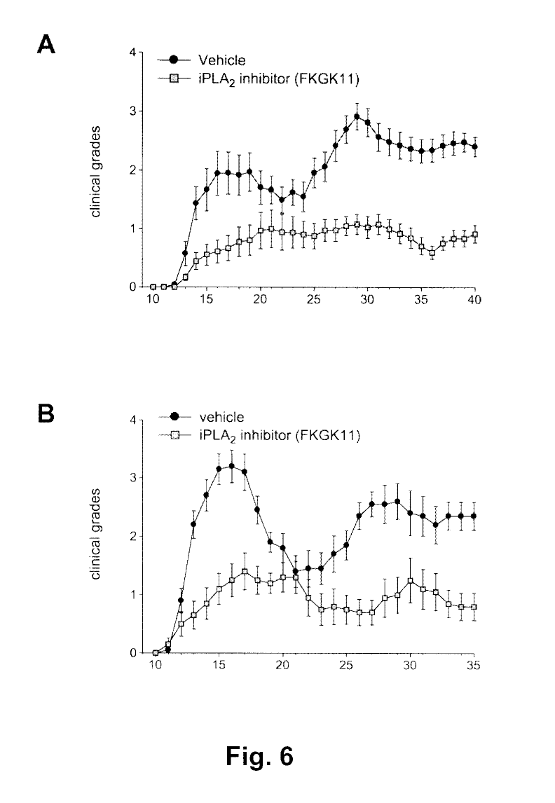

A) Treatment starting before onset of symptoms. To assess the effects of the fluoroketone compounds in the onset and progression of EAE, SJL/J mice in which EAE was induced by immunization with the myelin peptide, proteolipid protein, were used. Mice were treated with two fluoroketone compounds that selectively inhibit iPLA₂ (FKGK11), or cPLA₂/iPLA₂/sPL₂ (FKGK2) (see Table I). These inhibitors were given daily on a 3-day cycle (one intravenous injection followed by 2 intraperitoneal injections) for 3 weeks starting on day 6 after the immunization, i. e., before the onset of clinical symptoms. Mice were evaluated daily for clinical disability using the following 5-point scale: grade 0=normal; grade 1=flaccid tail; grade 2=mild hind limb weakness, quick righting reflex; grade 3=severe hind limb weakness, poor righting reflex; grade 4=hind limb paralysis; grade 5=hind limb paralysis and partial fore limb weakness. This analysis was done in a blinded fashion so the evaluator was unaware of the nature of the groups. Vehicle-treated animals began to develop symptoms by day 11, and reached the first peak of clinical attack at day 18 with an average maximum clinical score of about grade 2 (FIG. 6A). The symptoms then remitted between days 20 and 25, followed by a second clinical attack which reached an average clinical score of grade 3 around day 30 (FIG. 6A). This was followed by a slight remission and the animals showed a clinical deficit of grade 2.4 at day 40.

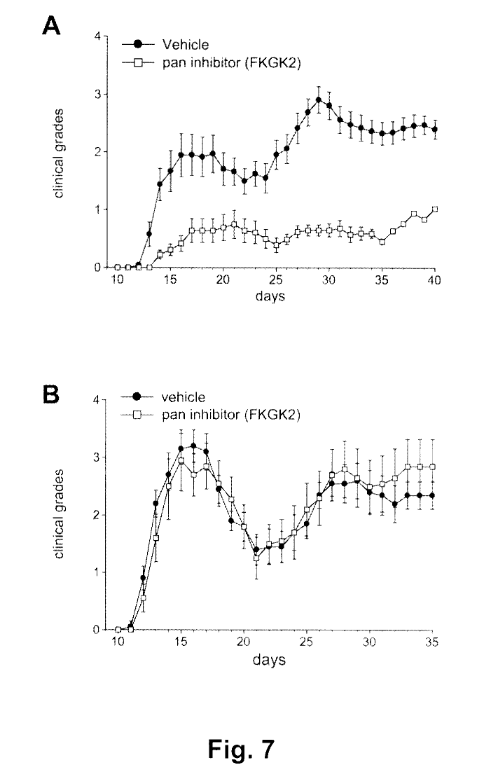

Mice treated with the iPLA₂-specific fluoroketone inhibitor FKGK11 showed marked reduction in the clinical severity and progression of EAE. These mice reached a maximum peak clinical disability score of only grade 1.0 throughout the course of disease until day 40 (FIG. 7A). The overall clinical profile shows slight dips in the scores that coincide with the times when the vehicle treated mice have periods of remission (FIG. 6A). These data suggest that iPLA₂ is involved in the onset and likely also in the subsequent progression of the disease. Mice treated with FKGK2, which blocks cPLA₂/iPLA₂/sPLA₂ had maximal average scores of around 0.5 for the major part of the clinical course of 40 days. This was similar statistically with the results with FKGK11 (FIG. 7A).

B) Treatment started after onset of symptoms (delayed treatment). When treatment with the fluoroketones was started after the onset of the clinical symptoms, i.e., day 11 after immunization and continued for 2 weeks, mice treated with FKGK11 showed a marked reduction in the severity and progression of the disease. The vehicle-treated mice showed a first clinical attack at day 15, with a peak score of 3.2 (FIG. 6B), followed by a second attack on days 26-29 with a score of 2.5. In contrast, animals treated with FKGK11 developed a maximal clinical disability score of only 1.4, on day 17 which then reduced to a score of 0.8 between days 22-27 and a slight second peak with a score of 1.2 on day 30 (FIG. 6B). These delayed treatment experiments suggest that iPLA₂ is involved in the progression of the disease after the initial onset of the disease. FKGK2-treated mice on the other hand did not show any significant improvement and displayed a comparable clinical severity and profile as the vehicle treated mice (FIG. 7B).

### Example 18

### Expression of Inflammatory Cytokines and Chemokines in the Spinal Cord of EAE Mice Following Treatment with PLA₂ Inhibitors

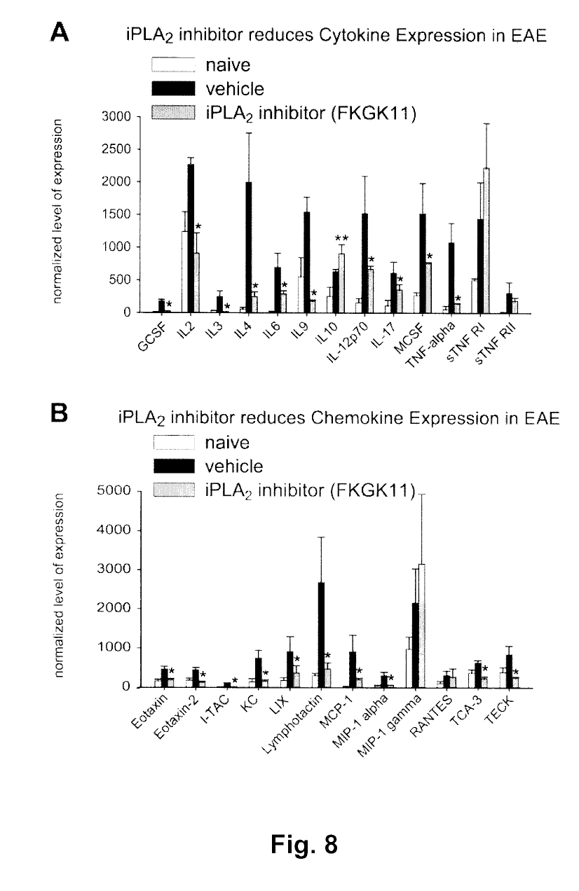

To further examine the effects of iPLA₂ inhibitor treatment, the expression of 40 inflammatory cytokines and chemokines was assessed using the RayBio® Mouse Inflammation Antibody Array 1.1 (RayBiotech Inc., Norcross, Ga, Cat. #0106008/AAM-INF-1). The analysis was carried out on spinal cords taken from the peak stage of clinical disability (day 18), from mice treated with either a vehicle control, or the iPLA₂-specific inhibitor FKGK11. The vehicle-treated control showed an increase of 17 cytokines and 15 chemokines (FIG. 8A, B), most of which are known to play a role in inflammation in EAE. Interestingly, the iPLA₂ inhibitor reduced the expression of 13 of these pro-inflammatory cytokines (FIG. 8A), while having an increase in IL-10, which is known to play an anti-inflammatory role in EAE. Also, there was a marked reduction in 11 of the chemokine proteins (FIG. 8B). This demonstrates that inhibiting the actions of iPLA₂ can prevent the robust inflammatory cascade seen in EAE.

### Example 19

### Expression of iPLA₂ After Spinal Cord Contusion Injury

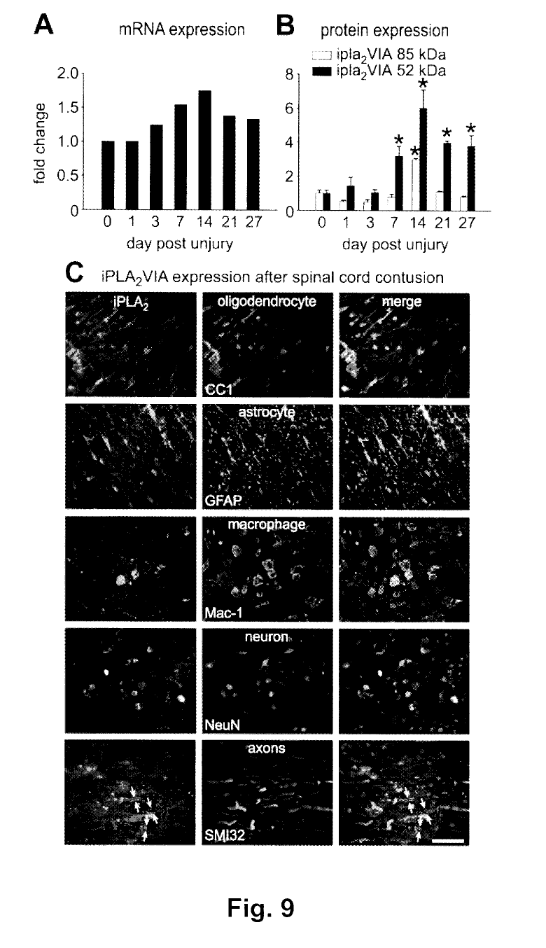

The expression of iPLA₂ mRNA after spinal cord contusion injury in mice was assessed. iPLA₂ (IVA) mRNA is increased after SCI above its constitutively expressed levels to reach a peak of ˜2-fold at 14 days post-injury (dpi) (FIG. 9A). Quantification of the protein expression detected by Western blotting showed significant elevation to 3.2-fold at 14 dpi. iPLA₂ has ankyrin-like repeats that negatively control the activity of the enzyme. Under conditions of stress, removal of these ankyrin-like repeats from the full length 85 kDa protein results in a smaller 52 kDa forms that has enhanced activity (Lauber K. et al. (2003). Cell 113: 717-730). Interestingly, a ˜50 kDa form of iPLA₂ was detected after SCI, which was significantly increased 6-fold from 7 to 28 dpi with a peak at 14 dpi (FIG. 9B). At 14 days, iPLA₂ was mainly expressed in oligodendrocytes at and adjacent to the lesion epicentre (FIG. 9C). Some astrocytes and a small population of Mac-1 positive macrophages in the lesion core, as well as some neurons located in the dorsal horns in areas adjacent to the lesion also expressed iPLA₂ (FIG. 9C). Interestingly, at 7 and 14 dpi, iPLA₂ immunostaining was also localized to axonal membranes of myelinated and unmyelinated axons (FIG. 9C). Increased iPLA₂ activity at these sites might therefore cause selective damage of axonal membranes and lead to axonal degeneration after SCI.

### Example 20

### Effect of iPLA₂ Inhibition after Spinal Cord Injury

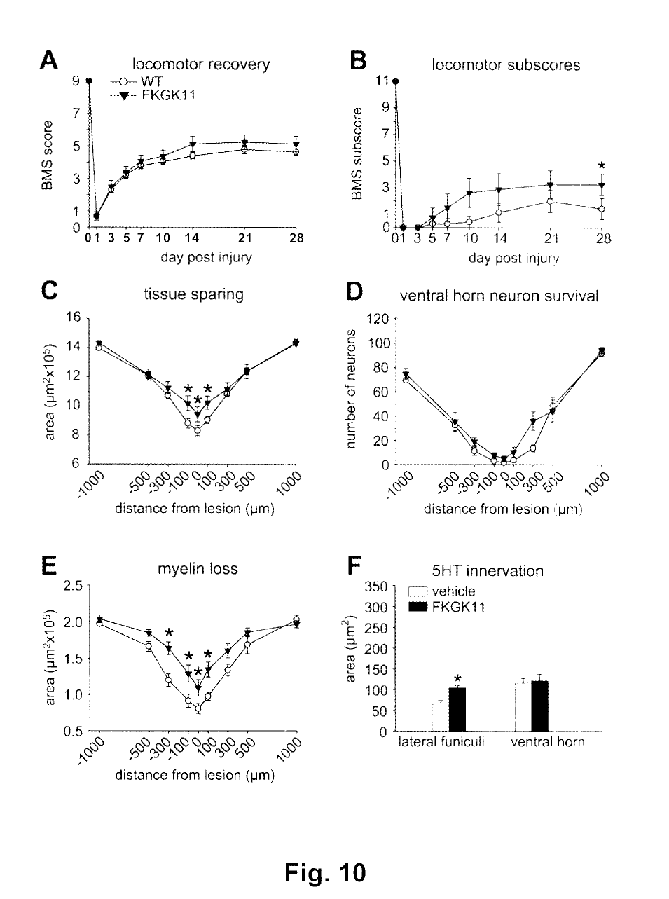

The role of iPLA₂ after SCI was assessed using a novel fluoroketone compound (FKGK11) that selectively blocks iPLA₂ activity (Table 1). Although consistent differences were seen in the main BMS scores (FIG. 10A), these differences did not reach statistical significance. However, iPLA₂ inhibitor treatment with FKGK11 led to a significant improvement in the BMS subscores, which rates fine locomotor coordination and control (FIG. 10B). Importantly, inhibiting iPLA₂ with FKGK11 also resulted in: (i) significantly greater tissue sparing (FIG. 10C); (ii) significant enhancement of myelin sparing in regions near the epicenter (FIG. 10E); and (iii) ˜40% increase in serotonergic (5-HT) fibers in the lateral white matter as compared to vehicle-treated SCI controls (FIG. 9F). The iPLA₂ inhibitor had little if any effect on neuronal survival (FIG. 9D). These data along with the expression data suggest that after SCI, iPLA₂ is involved in the damage of axons and myelin-producing cells.

TABLE I
Inhibition of PLA₂ by various perfluoroketone inhibitor
compounds described herein.

| | | GIVA | GVIA | |
|---|---|---|---|---|
| No | Structure | cPLA₂ | iPLA₂ | GV sPLA₂ |
| FKGK1 | 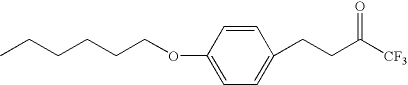 | 91 ± 2 X₁(50) 0.0199 ± 0.0025 | 85 ± 4 X₁(50) 0.0328 ± 0.0035 | 82 ± 8 |
| FKGK2 | 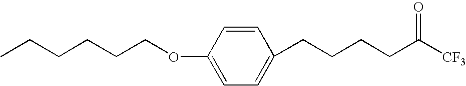 | 92 ± 3 X₁(50) 0.0098 ± 0.0006 | 91 ± 4 X₁(50) 0.0169 ± 0.0021 | 86 ± 2 |
| FKGK3 | 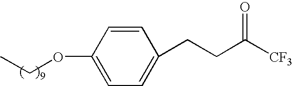 | 96 ± 2 X₁(50) 0.0156 ± 0.0019 | 94 ± 8 X₁(50) 0.0208 ± 0.0032 | 80 ± 6 |
| FKGK4 | 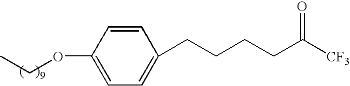 | 95 ± 2 X₁(50) 0.0116 ± 0.0012 | 94 ± 8 X₁(50) 0.0166 ± 0.0022 | 84 ± 7 |
| FKGK5 | 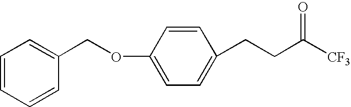 | 88 ± 1 | 71 ± 14 | 49 ± 12 |
| FKGK7 | 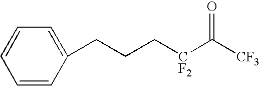 | 27 ± 3 | 49 ± 12 | 59 ± 12 |
| FKGK8 | 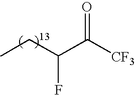 | 94 ± 2 X₁(50) 0.0167 ± 0.0018 | 93 ± 4 X₁(50) 0.0011 ± 0.0002 | 86 ± 10 X₁(50) 0.0236 ± 0.004 |
| FKGK11 | 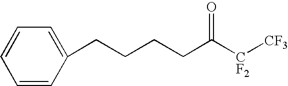 | N.D. | 98 ± 16 X₁(50) 0.0073 ± 0.0007 | 28 ± 1 |
| FKGK12 | 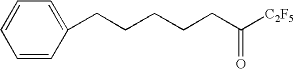 | 56 ± 4 | 98 ± 5 X₁(50) 0.0065 ± 0.001 | 46 ± 8 |
| FKGK13 | 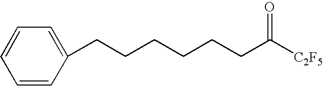 | 65 ± 12 | 98 ± 4 X₁(50) 0.0019 ± 0.0004 | 75 ± 10 |
| FKGK14 | 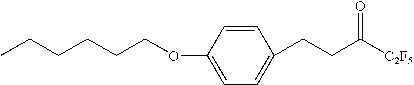 | 73 ± 4 | 95 ± 5 X₁(50) 0.0075 ± 0.0011 | 86 ± 4 |

Average percent inhibition and standard error (n = 3) reported for each compound at 0.091 mole fraction. X₁(50) values determined for inhibitors with greater than 90% inhibition. N.D. means compounds with less than 25% inhibition (or no detectable inhibition).

TABLE II
Perfluoroketone PLA₂ inhibitor compounds prepared in
the studies described herein.

| No | Structure |
|---|---|
| FKGK1 | 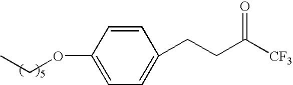 |
| FKGK2 | 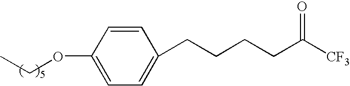 |
| FKGK3 | 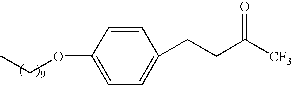 |
| FKGK4 | 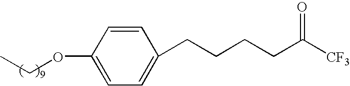 |
| FKGK5 | 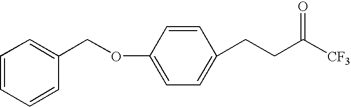 |
| FKGK6 | 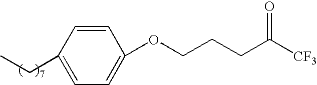 |
| FKGK7 | 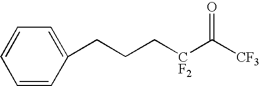 |
| FKGK8 | 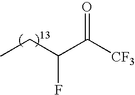 |
| FKGK9 | 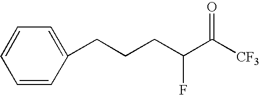 |
| FKGK10 | 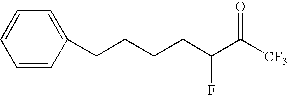 |
| FKGK11 | 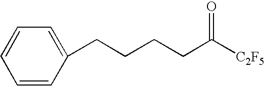 |
| FKGK12 | 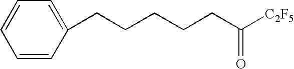 |
| FKGK13 | 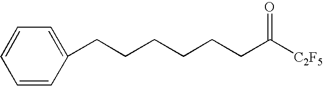 |
| FKGK14 | 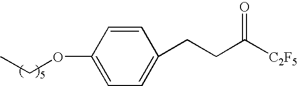 |
| FKGK15 | 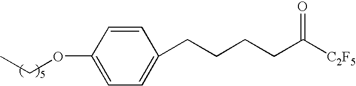 |
| FKGK16 | 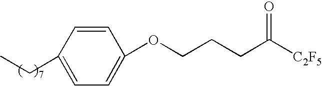 |
| FKGK17 | 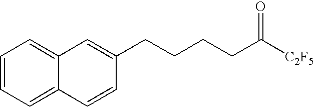 |
| FKGK18 | 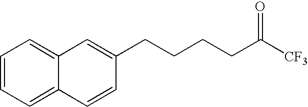 |
| FKGK19 | 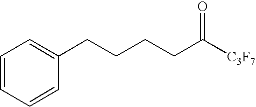 |
| FKGK20 | 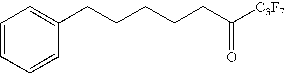 |
| FKGK21 | 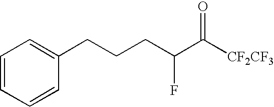 |
| FKGK22 | 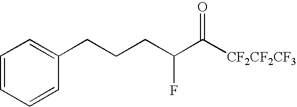 |

Although the present invention has been described hereinabove by way of specific embodiments thereof, it can be modified, without departing from the spirit and nature of the subject invention as defined in the appended claims.

1. A compound having the formula I or a hydrate thereof having the formula Ia: 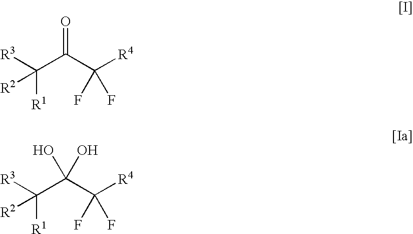
wherein;
R¹ is H, F or CH₃;
R² is H or F;
R³ is a alkyl, branched or linear, saturated or unsaturated; aryl, substituted or not; or heteroaryl, substituted or not;
R⁴is H, F, CF₃, (CF₂)ₖCF₃, 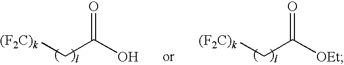
wherein k=0-4 and l=2-5;
or a pharmaceutically acceptable salt thereof.

2. The compound of claim 1, wherein R³ is: 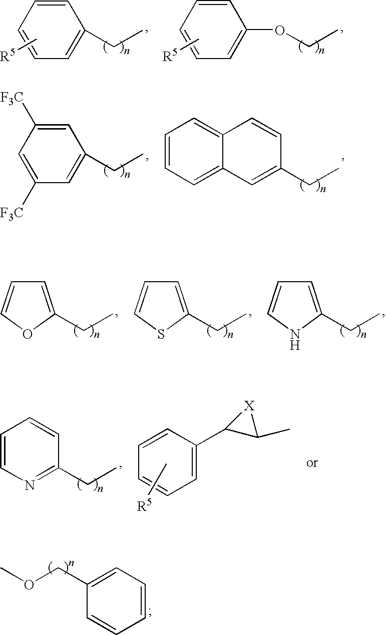
wherein
X=O, NH or S;
n=1-5 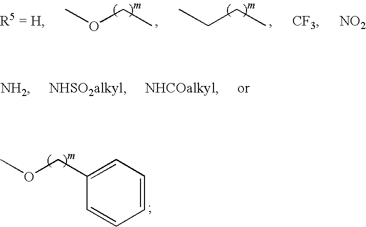
wherein m=1-9.

3. The compound of claim 1, wherein said alkyl is C₁-C₁₄ linear alkyl.

4. The compound of claim 3, wherein said alkyl is C₆-C₁₂ linear alkyl.

5. The compound of claim 1, wherein k=1 or 2.

6. The compound of claim 1, wherein said compound is: 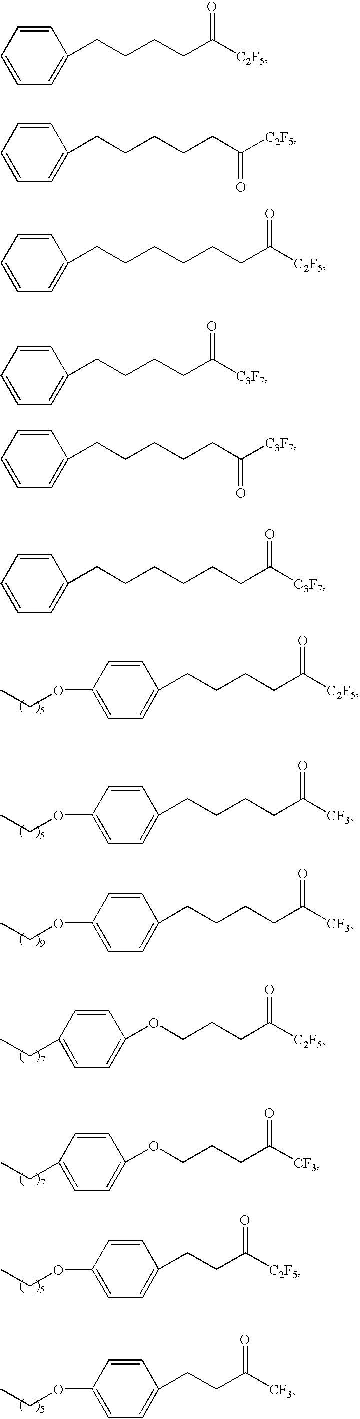 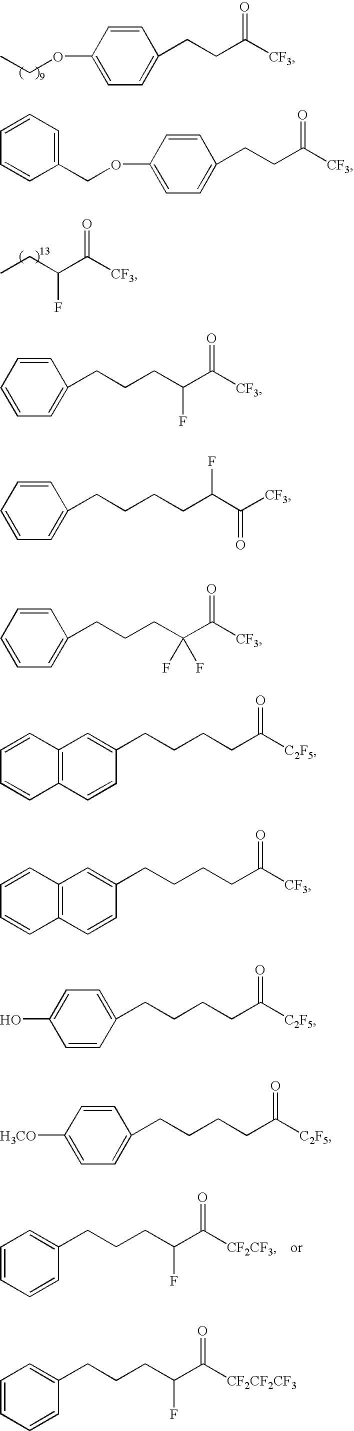
or a hydrate thereof, or a salt thereof.

7. A composition comprising the compound of claim 1 and a pharmaceutically acceptable carrier or excipient.

8-23. (canceled)

24. A method for inhibiting PLA₂ activity in a cell or subject, said method comprising contacting said cell with, or administering to said subject, an effective amount of the compound of claim 1.

25. A method for preventing or treating a neural disease or condition in a subject, said method comprising administering to said subject an effective amount of the compound of claim 1.

26. The method according to claim 25, wherein said neural disease or condition is an inflammatory disease or condition of the central nervous system.

27-29. (canceled)

30. The method according to claim 25, wherein said disease or condition is a demyelinating disease.

31. The method according to claim 30, wherein said demyelinating disease is multiple sclerosis.

32. The method according to claim 25, wherein said disease or condition is a neural injury.

33. The method according to claim 32, wherein said neural injury is spinal cord injury.

34. The method according to claim 25, wherein said subject is a mammal.

35. The method according to claim 34, wherein said mammal is a human.

36. A package or kit comprising the compound of claim 1 together with instructions for the prevention or treatment of a neural disease or condition, and/or for the prevention or treatment of an inflammatory disease or condition.

37-44. (canceled)

45. A method of preparing the perfluoroketone compound of formula I as defined in claim 1, the method comprising:
(a) converting a carboxylic acid of formula II 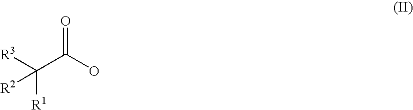
into a corresponding acyl chloride or acyl fluoride of formula III 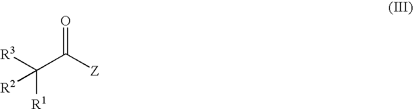
wherein Z=Cl or F; and
(b) reacting the compound of formula III with an anhydride of a perfluoroacid of formula IV 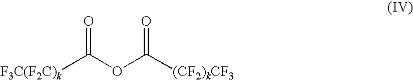
in the presence of an amine,
wherein k, R¹, R² and R³ are as defined in claim 1;
thereby obtaining the perfluoroketone compound of formula I.

46. A method of preparing the perfluoroketone compound of formula I as defined in claim 1, the method comprising:
(a) reacting a compound of formula (V), (VI) or (VII) 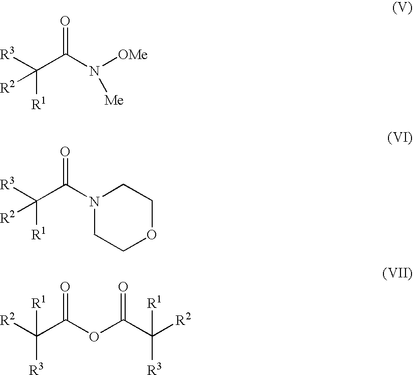
with a compound of formula (VIII)
I(CF₂)ₖCF₃ (VIII)
wherein k, R¹, R² and R³ are as defined in claim 1; and
(b) treating the reaction mixture of (a) with an organo-lithium reagent;
thereby obtaining the perfluoroketone compound of formula I.

47. A method of preparing the perfluoroketone compound of formula I as defined in claim 1, the method comprising:
(a) reacting an aldehyde of formula IX 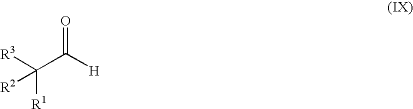
with a compound of formula VIII
I(CF₂)ₖCF₃ (VIII)
(b) treating the reaction mixture of (a) with an organo-lithium reagent to obtain a compound of formula X 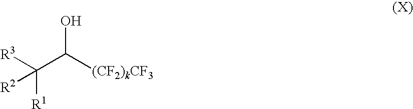
wherein k, R¹, R² and R³ are as defined in claim 1; and
(c) oxidizing the compound of formula X;
thereby obtaining the perfluoroketone compound of formula I.

48-49. (canceled)
